(19) United States
(12) Patent Application Publication
(10) Pub. No.: US 2024/0369544 A1
(43) Pub. Date: Nov. 7, 2024

(54) MULTIVALENT GLYCAN SCAFFOLDS AND METHODS OF USE THEREOF

(71) Applicant: NovAb, Inc., Tucker, GA (US)

(72) Inventors: Sylvain Daniel Lehoux, Lawrenceville, GA (US); Lovick Edward Cannon, Atlanta, GA (US); Mary Ellen Ballestas, Brookhaven, GA (US)

(21) Appl. No.: 18/686,779

(22) PCT Filed: Aug. 25, 2022
(86) PCT No.: PCT/US2022/075446

§ 371 Date: Feb. 26, 2024

Related U.S. Application Data
(60) Provisional application No. 63/237,068, filed on Aug. 25, 2021.

(51) Int. Cl. G01N 33/53

(52) U.S. Cl. CPC G01N 33/5308

(57) ABSTRACT

Multivalent glycan scaffolds, and methods of making, and compositions and methods of use thereof are provided. The multivalent glycan scaffolds are typically composed of an underlying branched scaffold such as a dendrimer, with two or more glycans and optionally one or more functional elements linked to the terminal ends thereof. The glycans can be synthetic or isolated from a natural source. Methods of using glycan scaffolds to identify binding partners such as cells and/or glycan binding proteins are also provided. A method of identifying glycan-based binding interactions can include contacting a target material with a known multivalent glycan scaffold and identifying element(s) from the target material that binds thereto. In other embodiments, a method of identifying glycan-based binding interactions includes contacting a known target material element with a library of different species of multivalent glycan scaffolds each having two or more glycans, and identifying glycan scaffold(s) that bind thereto.

### CROSS-REFERENCE TO RELATED APPLICATION

This application claims the benefit of and priority to U.S. Ser. No. 63/237,068 filed Aug. 25, 2021, and which is incorporated by reference in its entirety.

### REFERENCE TO SEQUENCE LISTING

The Sequence Listing submitted as an xml file named “NOVAB100PCTST25.xml,” created on Aug. 22, 2022, and having a size of 6,353 bytes, is hereby incorporated by reference pursuant to 37 C.F.R § 1.834(c)(1).

### FIELD OF THE INVENTION

This invention is generally in the field of glycan binding, and compositions and methods of use related thereto.

### BACKGROUND OF THE INVENTION

Primarily attached to lipids or proteins, glycans are abundantly expressed on surfaces of all cells. By their immense structural and compositional diversity glycans play a major role in numerous biological processes among all living organisms from viruses and bacteria to plants and animals. The proteins that bind glycans, known as glycan-binding proteins (GBPs), are typically expressed on the surface of cells and mediate various cell signaling. CIS- or TRANS-binding interaction of a cell surface glycan ligand with its corresponding cell surface GBP is therefore a multivalent interaction since many copies, i.e., hundreds to tens of thousands identical copies, of each distinct glycan structure and each distinct GBP will be present on cell surfaces. Such multivalent interactions result in a substantial increase in apparent binding affinity (avidity) due to “forced proximity” (Vauquelin, et al., Br J Pharmacol., 168(8):1771-85, doi: 10.1111/bph.12106 (2013)); the high local “target” concentrations experienced by unbound glycan ligand or GBP once one (or more) glycan ligand(s) has bound to complementary GBP(s). Forced proximity significantly increases binding/rebinding of glycan:GBP complexes and substantially slows dissociation of the interaction, since all glycan:GBP complexes must dissociate simultaneously. This multivalent avidity enhancement increases apparent affinity many thousands-fold versus monovalent binding and accounts for the difficulty with inhibiting glycan:GBP-mediated cell-cell interactions despite the poor monovalent affinities that typify glycan:GBP binding.

Discovering and characterizing glycan binding molecules, including lectins, antibodies and other carbohydrate binding biomolecules, requires appropriate glycan epitope bait counterparts. Because monovalent interactions between glycans and glycan binding molecules are usually weak, with a low affinity and a K_{d} in the millimolar to micromolar range, multivalent presentation of glycans is important to increase avidity and apparent affinity to enable capture of the GBP-glycan complex or effectively compete with multivalent GBP-glycan interactions.

The commercially availability of glycans presented in a multivalent format are very limited. Such glycans are usually coupled to a polymer (such as Poly Acrylic Acid (PAA)) and are mostly restricted to structures consisting of 1 to 4 monosaccharide units, only representing common glycan epitopes (blood groups, Lewis structures) that are relatively easy to synthesize or purify from natural sources.

Multivalent presentation of glycans using a wide range of scaffolding constructs including gold nanoparticles, fullerene multivalent scaffolds, carbon nanotubes, glycodendrinanoparticles using self-assembled peptides, glycan oligonucleotides, peptide/protein scaffoldings, and glycan scaffolds have been investigated (Spinelli, et al., Chem Soc Rev., 42(11):4557-73, doi: 10.1039/c2cs35406c (2013); Reichardt, et al., Chem Soc Rev., 42(10):4358-76, doi: 10.1039/c2cs35427f (2013); Bernardi, et al., Chem Soc Rev., 42(11):4709-27, doi: 10.1039/c2cs35408j (2013); Muller, et al., Chem Soc Rev., 45(11):3275-302, doi: 10.1039/c6cs00165c (2016). Despite using various synthetic strategies, these materials only partially address the issue, as the majority of multivalent glycan constructs reported in the literature only displayed mono- or disaccharides (Al-Mughaid,et al., J Org Chem., 71(4):1390-8, doi: 10.1021/jo052045u (2006); Chabre, et al., J Org Chem., 73(14):5602-5, doi: 10.1021/jo8008935 (2008); Martinez-Avila, et al., Chemistry, 15(38):9874-88, doi: 10.1002/chem.200900923 (2009); Ribeiro-Viana, et al., Nat Commun., 3:1303, doi: 10.1038/ncomms2302 (2012); Munoz, et al., Nat Chem., 8(1):50-7, doi: 10.1038/nchem.2387 (2016); Budhadev et al., J Am Chem Soc., 142(42):18022-18034, doi: 10.1021/jacs.0c06793 (2020); Ren, et al., Talanta., 223(Pt 1):121706, doi: 10.1016/j.talanta.2020.121706 (2021)). A few other “glycocluster” builds used derivatives of the bi-antennary N-glycans found in chicken egg yolk SialoGlycoPeptide (SGP) (Tanaka, et al., Angew Chem Int Ed Engl., 49(44): 8195-8200, doi:10.1002/anie.201000892 (2010); Smirnov, et al., Small., 16(46):e2004831, doi: 10.1002/smll.202004831 (2020)). However, none of these strategies offer a simple and universal method suitable to most glycans and designed to build multivalent glycan polymers, and larger structures, including full O-linked and N-linked glycans, are not available as multivalent polymers, or simply not available in any format.

Thus, it is an object of the invention to provide alternative and improve means of presenting glycans in a multivalent format.

### SUMMARY OF THE INVENTION

Multivalent glycan scaffolds, and methods of making, and compositions and methods of use thereof are provided. The multivalent glycan scaffolds are typically composed of an underlying branched scaffold with two or more glycans and optionally one or more functional elements linked to its terminal ends. The underlying branched scaffold can be, for example, a dendrimer or another branched polymer. The glycans can be synthetic, but more preferably are isolated from a natural source. Each scaffold is typically functionalized with 2, 3, 4, 5, 6, 7, 8, 9, 10, or more glycans which can be single species of glycan (i.e., homogeneous) or a mixture of two or more different species of glycan (i.e., heterogenous). Functional element(s) can be, for example, an agent that facilitates formation, imaging, localization, identification, isolation, or characterization of the glycan scaffold, or a binding partner thereof. Exemplary functional elements include, but are not limited to, a nucleic acid having a unique barcode sequence that specifically identifies the glycan attached to the scaffold, a fluorophore, e.g., to facilitate formation, detection, imaging, localization, or sorting of the scaffold, and an affinity tag that can be used to isolate or immobilize the scaffold. In exemplary, non-limiting embodiment, the glycan is linked to 2-amino-N-(2-aminoethyl)-benzamide (AEAB) which is directly or indirectly linked to the scaffold. In some embodiments, the glycans are linked to the scaffold directly or indirectly through an amino acid, which may be alone or form part of a polypeptide chain. Thus, in some embodiments, the glycan scaffold is a glycopeptide scaffold. In some embodiments, a single polypeptide chain links and/or displays two or more glycans, which can be the same or different. Exemplary amino acids include, but are not limited to, serine and threonine.

Methods of using the glycan scaffolds to identify binding partners such as cells and/or glycan binding proteins (GBPs) are also provided. In some embodiments, a method of identifying glycan-based binding interactions includes contacting a target material with a known multivalent glycan scaffold and identifying element(s) from the target material that binds thereto. The target material can be, for example, a population of cells having one, two, or more different cell types, or a cell lysate or other proteinaceous mixture having one, two, or more GBP. The methods can also be used to determine whether a specific cell type expresses a GBP(s) that binds a specific glycan. When the target material is a population of cells, the target material element is typically one or more of the cell types therein. Preferably, the one or more cell types preferentially bind to the multivalent glycan scaffold compared to other cell type(s) in the population. Further steps can include isolating the multivalent scaffold with the cells bound thereto and/or characterizing the bound cells. When the target material is a cell lysate or other proteinaceous mixture, the target material element is typically one or more of the GBPs therein. Preferably, the one or more GBPs preferentially bind to the multivalent glycan scaffold compared to other GBPs in the cell lysate or other proteinaceous mixture. Further steps can include isolating and/or characterizing the bound GBP.

In other embodiments, a method of identifying glycan-based binding interactions includes contacting a known target material element with a library of different species of multivalent glycan scaffolds each having two or more glycans, and identifying glycan scaffold(s) that bind thereto. The library may contain, for example, one or a plurality of copies of between about 2 about 10,000 or more different species of glycan scaffolds, or any subrange or specific integer thereof, such as 2, 5, 10, 25, 50, 75,100,250, 500, 1,000. The known target material element can be a cell. Preferably, one or more of the multivalent glycan scaffolds preferentially bind to the cell compared to other multivalent glycan scaffolds in the library. In this way, the compositions and methods can be used to identify the glycan specifity(ies) of cellular GBPs. In other embodiments, the known target material element is a GBP. Preferably, one or more of the multivalent glycan scaffolds preferentially bind to the GBP compared to other multivalent glycan scaffolds in the library. Further steps can include isolating and/or characterizing the multivalent glycan scaffold(s).

Glycan binding interactions identified according to the provided methods can be used to rationally design multivalent glycan scaffolds for use in therapeutic and diagnostic applications. For example, a method of treating a subject in need thereof can include identifying a glycan-based binding interaction according to any of the disclosed methods and administering the subject a composition including an effective amount of a therapeutic multivalent glycan scaffold having the glycan of the identified interaction. Preferably, the scaffolds preferentially bind to target cells and/or GBP in the subject. In some embodiments, therapeutic glycan scaffolds induce or inhibit a cellular activity upon binding to the target cell or GBP of the interaction. In some embodiments, therapeutic glycan scaffolds include an active agent functionalized thereto. The active agent can be a nucleic acid, small molecule, polypeptide, etc. For example, in some embodiments, the target cell is a cancer cell and the active agent is an anti-cancer drug. In other embodiments, the target cell is an immune cell and the active agent is or encodes an antigen. Similarly, glycan scaffolds functionalized with an imaging agent can be used to identify, track, or monitor target cells or GBP and can be utilized in diagnostic applications.

Compositions including the glycan scaffolds are also provided. In some embodiments, the compositions include glycan scaffolds that are bound to cells or GBP. The glycan scaffolds, with or without their binding partners, can be immobilized, optionally to a surface or substrate such as bead, a slide, a plate, or a dish, or they can be free in solution, such as media or a pharmaceutical composition.

### BRIEF DESCRIPTION OF THE DRAWINGS

FIG. 1A is a flow diagram illustrating how exemplary glycans containing a reducing end can be obtained commercially (e.g., H-type 2-trisaccharide (Fucα1-3Galβ1-4GlcNAc)), released, and purified from natural sources (e.g., by PNGaseF), or by non-reducing alkaline β-elimination. FIG. 1B is an illustration of components of an exemplary multimeric ligand of the disclosed technology. The scaffold determines the ligand valency and size of the structure. Detection tag(s) facilitate detection and capture of the structure. The optional barcode can provide a unique DNA or RNA sequence associated with each distinct ligand present in a library of multimeric ligand structures. FIG. 1C is a flow diagram of a library of multimeric ligand particles (MLPs) each presenting a distinct ligand and DNA or RNA sequence identifier that identifies ligands that target specific cells and/or tissues.

FIGS. 2A and 2B are flow diagrams illustrating the generation of 113 glycan dendrimers of Example 1. FIG. 2A illustrates the azidation of 113 reducing glycan. FIG. 2B illustrates a click chemistry reaction to couple H3-N3 to 8-mer PEG Biotin/Alkyne dendrimers. FIG. 2C is a bar graph showing the results of a flow cytometry binding experiment with O13 YSD cells (yeast cells expressing the lamprey O13 VLRB anti-H3 antibody on their surface) and multivalent H3-PAA biotin, monovalent H3-sp biotin, multivalent H3 glycan dendrimer biotin and unmodified dendrimer biotin. Only multivalent H3-PAA and H3 glycan dendrimer were effectively bound by O13 YSD cells. FIG. 2D is a bar graph showing the results of an ELISA binding experiment with O13-human IgG1 Fc fusion protein (coating anti-H3 antibody) and multivalent H3-PAA biotin, multivalent H3 glycan dendrimer biotin, unmodified dendrimer biotin and multivalent LewisA-PAA. Multivalent H3-PAA and 113 glycan dendrimer are comparatively bound by O13-hIgG antibody whereas the unmodified dendrimer and the LewisA-PAA compounds are not bound. FIGS. 2E-2G are directed to the generation and testing of 2nd batch of H3-dendrimers. FIG. 2E is a bar graph showing PD10 fractions assayed by O13-hIgG anti-H3 antibody. FIG. 2F is a plot showing H3-N3-magnetic beads detected by O13-hIgG antibody. FIG. 2G is a bar graph showing H3-dendrimer-magnetic beads detected by ConA and AAL lectins.

FIGS. 3A-3D illustrate generation and testing of Man5 and Man9 glycan dendrimers. FIG. 3A illustrates Man5- and Man9-AEAB compounds (NatGlycan). FIGS. 3B and 3C are flow diagrams illustrating a conjugation reaction to PEG-NHS to generate Man5 and Man9 dendrimers. FIG. 3D ConA and PNA lectin reactivity to unmodified PEG-NHS, Man5 and Man9 dendrimers.

FIGS. 4A-4D illustrate generation and testing of Core1-T, Core2-S, Core3-S and Core4-S glycan dendrimers (T=threonine, S=serine). FIG. 4A is an illustration of the structures of Core1-tT, Core2,3,4-S(Chemily Glycoscience). FIG. 4B is a flow diagram of a conjugation reaction with PEG-NHS to generate of Core1-T, Core2,3,4-S dendrimers (Core2-S as an example). FIG. 4C is a bar graph showing ConA, PNA and GSLII lectin reactivity to unmodified PEG-NHS, Core1-T and Core2-S dendrimers. FIG. 4D is a bar graph showing PNA and GSLII lectin reactivity to unmodified PEG-NHS, Core3-S and Core4-S dendrimers.

FIGS. 5A-5D show the generation and testing of GP100121 (SEQ ID NO:5) glycopeptide dendrimers. FIG. 5A illustrates a synthetic glycopeptide (SEQ ID NO:5) containing 2 GalNac linked to 1 serine and 1 threonine (GP100120, Sussex Research). FIGS. 5B and 5C are flow diagrams of conjugation reaction to PEG-NHS to generate GP100121 (SEQ ID NO:5) glycopeptide dendrimers. FIG. 5D is a bar graph showing PNA and VVL lectin reactivity to unmodified PEG-NHS and GP100121 dendrimer.

### DETAILED DESCRIPTION OF THE INVENTION

### I. Definitions

As used herein, a “dendrimer” is a substance composed of identical dendrimer molecules.

As used herein, a “dendrimer molecule” is a molecule consisting of one or more dendrons emanating from a single constitutional unit.

As used herein, a “dendron” is part of a molecule with only one free valence, having exclusively dendritic and terminal constitutional repeating units (CRU) and in which each path from the free valence to any end-group comprises the same number of constitutional repeating units. For the purpose of determining the nature of constitutional repeating units the free valence is treated as a connection to a CRU. A dendrimer molecule having only one dendron is sometimes referred to as a dendron, monodendron or functionalized dendron. In a dendron, macrocycles of constitutional units are absent.

As used herein, a “glycan” is the carbohydrate portion of a glycoconjugate, such as a glycoprotein, glycolipid, or a proteoglycan. “Glycans” also refers to a free, unbound carbohydrates in biological media. Glycans can be singular monosaccharides and/or two or more monosaccharides linked glycosidically (e.g., polysaccharides).

As used herein, “active agent” refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder.

As used herein, “effective amount” and “suitable amount” with respect to a therapeutic agent is at least the minimum concentration required to effect a measurable improvement or prevention of any symptom or a particular condition or disorder, to effect a measurable enhancement of life expectancy, or to generally improve patient quality of life. The effective amount may vary depending on such factors as the disease or condition being treated, the active agent(s) (e.g., particular targeted constructs, etc.) being administered, the size of the subject, or the severity of the disease or condition. With regard to cancer, an effective amount can refer to an amount of the active agent that reduces or inhibits tumor growth or tumor burden. The effective amount can be in the context of the delivery systems disclosed herein. For example, in some embodiments, “therapeutically effective amount” refers to an amount of the therapeutic agent that, when incorporated into and/or onto particles described herein, produces some desired effect at a reasonable benefit/risk ratio applicable to any medical treatment. One of ordinary skill in the art may empirically determine the effective amount of a particular compound without necessitating undue experimentation.

As used herein, “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “antibody” refers to natural or synthetic antibodies that bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that bind the target antigen. “Single chain Fv” and “scFv” as used herein means a single chain variable fragment that includes a light chain variable region (V_{L}) and a heavy chain variable region (V_{H}) in a single polypeptide chain joined by a linker which enables the scFv to form the desired structure for antigen binding (i.e., for the V_{H} and V_{L} of the single polypeptide chain to associate with one another to form a Fv). The V_{L} and V_{H} regions may be derived from the parent antibody or may be chemically or recombinantly synthesized.

As used herein, “individual,” “host,” “subject,” and “patient” are used interchangeably to refer to any individual who is the target of administration or treatment. The subject can be a vertebrate, for example, a mammal. The subject can be a human or veterinary patient.

As used herein, “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### II. Compositions

Multimeric, multifunctional glycan scaffolds and methods of use thereof are provided. The scaffolds are typically spherical, branched or highly branched polymer that can mimic mammalian cells and create a high-density multivalent glycan for a native protein-glycan binding event. In addition to displaying multimeric glycans, the scaffolds can be optionally functionalized with a second or more moiety or element that allows for characterization and/or isolation of the native binding event. In preferred embodiments, the multimeric glycan scaffolds bind to surface of cells or glycan binding protein (GBP) and facilitate the characterization and/or isolation of bound cells or protein. Thus, mixtures of multifunctional glycan scaffolds and cells or protein, cells or protein with multifunctional glycan scaffold bound thereto are also provided. Each of these elements is discussed in more detail below.

### A. Multivalent Glycan Scaffolds

Multimeric glycan scaffolds are provided. The glycan scaffolds are typically formed of an underlying branched scaffold functionalized on its terminals ends with one or more glycans and optionally one or more other functional elements.

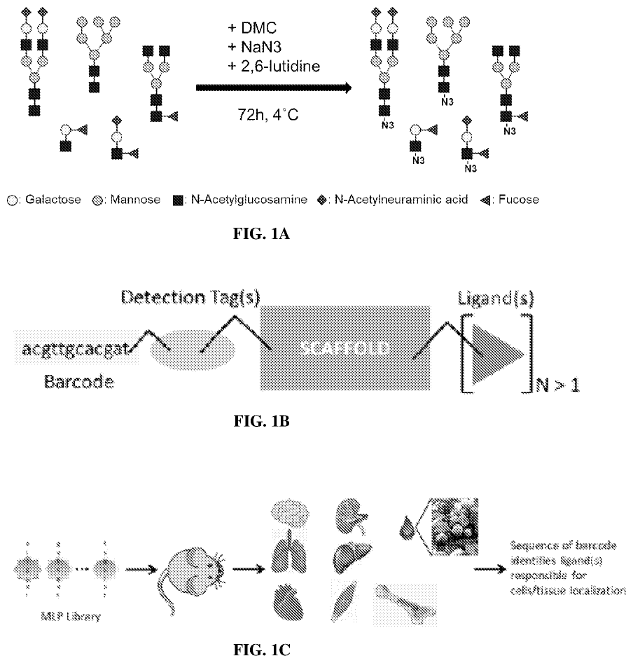

An example of a functionalized multivalent glycan scaffold is illustrated in FIG. 1B. FIG. 1B illustrates an embodiment of the formula:

(Ligand)n:Scaffold:Detection/Separation Tag:Identifier Tag
wherein “Ligand” is one or more species of glycan having n >1 copies (i.e., multivalent);
“Scaffold” is the underlying branched scaffold, e.g., dendrimer or hyperbranched polymer;
“Detection/Separation” and “Identifier” “Tag(s)” are exemplary, optional, functional elements e.g., (a) biotin or (HIS)6-8 peptide tag to facilitate separation/isolation, (b) a fluorophore to facilitate detection, and/or (c) a unique DNA or RNA sequence (“barcode”) that can be PCR amplified and sequenced to facilitate identification.

Alternatively, the “Tag(s)” of the formula can be an functional element(s) such as drug or other material(s) as discussed elsewhere herein, e.g., nucleic acids such as DNA, RNA, iRNA, crispr guide RNA, etc., or vesicle containing a drug or such other materials that are intended to have a therapeutic or cell modifying effect or can include an antigen that is intended to induce (a) a protective immune response directed to a pathogen or an aberrant cell, e.g., tumor, that expresses the antigen or (b) immune tolerance to the antigen as a therapy for an autoimmune disorder directed against the antigen.

Each of these elements of the multivalent scaffolds are discussed in more detail below.

### 1. Underlying Scaffold

The materials for forming underlying scaffolds are provided. The underlying scaffolds are typically branched scaffold formed of dendritic molecule. The field of dendritic molecules can be roughly divided into low-molecular weight and high-molecular weight species. The first category includes dendrimers and dendrons, and the latter includes dendronized polymers, hyperbranched polymers, and the polymer brush. Dendrimers and dendrons are monodisperse and usually highly symmetric, spherical compounds. The underlying scaffold can be formed of any one of these dendritic molecules.

The properties of dendrimers are controlled in-part by the functional groups on the molecular surface, however, there are examples of dendrimers with internal functionality. Controllable properties of dendrimers include solubility, toxicity, crystallinity, tecto-dendrimer formation, and chirality. See also Cloninger, Curr Opin Chem Biol., 6(6):742-8, doi: 10.1016/s1367-5931(02)00400-3 (2002).

Dendrimers can be considered to have three major portions: a core, an inner shell, and an outer shell, and can be synthesized to have different functionality in each of these portions to control properties such as solubility, thermal stability, and attachment of compounds. Synthetic processes can also precisely control the size and number of branches on the dendrimer.

Preparation of dendrimers requires a level of synthetic control achieved through series of stepwise reactions comprising building the dendrimer by each consecutive group. Dendrimer synthesis can be of the convergent or divergent type. During divergent dendrimer synthesis, the molecule is assembled from the core to the periphery in a stepwise process involving attaching one generation to the previous and then changing functional groups for the next stage of reaction. Functional group transformation is necessary to prevent uncontrolled polymerization. Such polymerization would lead to a highly branched molecule that is not monodisperse and is otherwise known as a hyperbranched polymer. Due to steric effects, continuing to react dendrimer repeat units leads to a sphere shaped or globular molecule, until steric overcrowding prevents complete reaction at a specific generation and destroys the molecule's monodispersity. The underlying branched scaffold of the disclosed multivalent glycan scaffolds can be prepared by convergent or divergent synthesis. The underlying scaffolds can be monodisperse or polydisperse.

The underlying scaffold is typically nanometer size, for example between 100 nm and 1 nm inclusive, or an integer subrange thereof or discrete integer therebetween. For example, the molecule may be about 100 nm, 75 nm, 50 nm, 25 nm, 20 nm, 15 nm, 12.5 nm, 10 nm, 7.5 nm, 5 nm, 4 nm, 3 nm, or 2 nm in diameter.

Dendrimers can be classified by generation, which refers to the number of repeated branching cycles that are performed during its synthesis. For example, if a dendrimer is made by convergent synthesis, and the branching reactions are performed onto the core molecule three times, the resulting dendrimer is considered a third generation dendrimer. Each successive generation results in a dendrimer roughly twice the molecular weight of the previous generation. Higher generation dendrimers also have more exposed functional groups on the surface, which can later be used to customize the dendrimer for a given application.

The underlying branched scaffold of the disclosed multivalent glycan scaffolds can be of any generation. Thus, in some embodiments, the dendrimers or other branched molecules can be of, for example, any one of G1-G10 generation. In some embodiments, the underlying branched scaffold has 1, 2, 3, 4, 5, 6, 7, 8, 9, or 10 repeating units, or any range derivable therefrom. In some embodiments, the underlying branched scaffold used herein are G0, G1, G2, or G3. However, the number of possible generations (such as 11, 12, 13, 14, 15, 20, or 25) may be increased by reducing the spacing units in the branching polymer.

Non-limiting examples of materials that can be used to form the underlying scaffold include, but are not limited to, polyamidoamine (PAMAM) dendrimer, a multi-arm polyethylene glycol (PEG), a branched polyethylenimine (PEI) polymer such as poly(propyleneimine), a PEG-core dendrimer, a multi-arm polyethylene glycol (PEG) polymer, a poly(acrylate), a polyamine, a polyamide, a polyether, a polyester, a poly(methyl acrylate), a polyphenylenes, and a polystyrene.

For example, in some embodiments, dendrons of the underlying scaffold are formed from polyester-8-hydroxyl-1-acetylene bis-MPA dendron, polyester-6-hydroxyl-1-acetylene bis-MPA dendron, polyester-32-hydroxyl-1-acetylene bis-MPA dendron, polyester-8-hydroxyl-1-carboxyl bis-MPA dendron, polyester-6-hydroxyl-1-carboxyl bis-MPA dendron, or polyester-32-hydroxyl-1-carboxyl bis-MPA.

In some examples, the underlying branched scaffold is formed of a hyperbranched polymer that forms polydisperse dendritic macromolecules having dendrimer-like properties. Often, hyberbranched polymers are prepared in a single synthetic polymerization step. For example, the hyperbranched polymer may be based on 2,2-bis(hydroxymethyl)propanoic acid (bis-MPA) monomer. Examples of hyperbranched polymers include, but are not limited to, hyperbranched bis-MPA polyester-16-hydroxyl, hyperbranched bis-MPA polyester-32-hydroxyl, and hyperbranched bis-MPA polyester-64-hydroxyl.

Peptidic and polysacharide “arms” can also be used to form the underlying scaffold.

In some embodiments, the branched polymer is a polyamidoamine (PAMAM) dendrimer having a number of surface groups (valency sites) in the range from 4, for Generation 0, to 4,096, for Generation 10, or any integer subrange or specific integer thereof. In other embodiments, the branched polymer is a multi-arm polyethylene glycol (PEG) polymer having a number of valency sites in the range from 2, for Y-shaped PEG, to 8, for 8-armed PEG. In other embodiments, the branched polymer includes a highly branched polyethylenimine (PEI) polymer having a number of valency sites in the range 10, for a 800 Da low-molecular-weight PEI, to 211, for a 25,000 Da high-molecular-weight PEI or any integer subrange or specific integer thereof.

The arm length can be customized for different spacings. For example, in particular embodiments, the scaffold has PEG length per arm of 1-50 PEG units, more preferably 5-25 PEG units.

In another example, the branched polymer is Dendro Alkyne-PEG-Dendro Alkyne (PEG Hyperbranched Dendrimer Acetylene) (e.g., G4, G5, or G6; PEG 10K). See, Creative PEGWorks, catalogue numbers HBP-211, HBP-212, and HBP-213.

In particular embodiments, the multimeric glycan scaffolds are between 10 kDa and 50 kD, or between 20 kDa and 60 kDa, or between 20 kDa and 30 kDa, each inclusive, or any integer range thereof, or any specific integer therebetween, optionally with 8 to 16 arms, e.g., PEG arms. These scaffolds are believed to provide enough/adequate spacing and flexibility for multimeric interactions. In some embodiments, the underlying scaffold is larger and/or wider. Larger/wider glycan scaffolds would potentially provide interactions for multiple GBPs at once, which may or may not be desirable, depending on the application for which it is being used.

Preferably, multimeric glycan scaffolds are soluble in water. Preferably, the glycan scaffolds are stable and/or nontoxic, particularly for in vivo applications. For example, when the underlying scaffold is composed of PEG, the multimeric scaffolds are highly water soluble because both PEG and glycans are very soluble in water and aqueous buffers.

### 2. Linkers and Linkages

The disclosed multimeric glycan scaffolds have two or more glycans and can optionally but preferably also have one or more functional elements presented at the terminal ends of the underlying scaffold. Thus, the underlying scaffold is typically manufactured with one or more terminal group(s) suitable for linking the glycans and optional additional functional elements. Such group(s) can be referred to a linker or linkage.

The underlying scaffold may be attached to the glycan or functional element through one or more bifunctional linkers that react with a terminal group on another bifunctional linker, or the underlying scaffold. Thus, in some embodiments, the linker is a bifunctional linker that tethers a terminal end of the underlying scaffold to a glycan or functional element through independent functional groups. The number of bifunctional linkers may be one or more. In some embodiments, there is a single bifunctional linker between the underlying scaffold and the glycan or functional element. In other embodiments, there may be two or more bifunctional linkers between the underlying scaffold and the glycan or functional, for example, an underlying scaffold having one end bonded with one end of a first bifunctional linker with the other end of the first bifunctional linker attached to one end of a second bifunctional linker. The second bifunctional linker may be attached through one functional end to the first bifunctional linker and the other functional end to the glycan. In this manner, there may be three, four or more bifunctional linkers between the underlying scaffold and the glycan or functional element. The number of bifunctional linkers may be chosen, for example, to provide more favorable steric properties, for more efficient linking or to take advantage of commercially available linkers, for example.

The underlying scaffold can include, but need not be limited to, at least one terminal group which can include a sodium carboxylate terminal group, a primary amine terminal group, a hydroxyl terminal group, an amidoethanol terminal group, a succinamic acid terminal group, a succinamidyl terminal group, and a mixture of primary and secondary amine terminal group. Where the underlying scaffold includes a terminal amine group, amine-reactive crosslinker groups may be used, which bind to primary amines. Groups that are capable of bonding with primary amines include isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these groups conjugate to amines by either acylation or alkylation.

Glycans may generally be attached through, for example, an amine group where the glycan includes an amine group, or through an aldehyde or ketone where the glycan includes a reducing sugar. For crosslinking to a glycan that includes a reducing sugar, a functional group reactive with aldehydes or ketones as appropriate will act to crosslink to the glycan. Aldehyde-reactive crosslinking functional groups include hydrazides to form hydrazone bonds. Aniline can be used to catalyze the hydrazide-aldehyde reaction. Another functional group includes an alkoxyamine group, where the alkoxyamine reacts with the aldehyde to form the stable oxime. Where the glycan includes an amine group, amine-reactive crosslinker groups may be used, which bind to primary amines, as discussed above.

Thus, the link between the underlying scaffold and the glycan or functional element can occur through crosslinking with a moiety which is a bifunctional linker with a first functional end to attach to the glycan or function moiety and a second functional end to attach to the underlying scaffold. In one embodiment, the linker or bifunctional linker includes one or more functional groups which are succinimidyl, hydrazidyl, aminooxyl, aldehyde, amino, azidyl, biotinyl, carboxyl, epoxy, hydrazinyl, N-hydroxysuccinimidyl, and ketonyl functional groups, having a spacer between the functional ends.

For example, in other more specific embodiments, the bifunctional linker includes a first succinimidyl homo-bifunctional linker which can be one or more of N,N′-disuccinimidyl carbonate (DSC), N, N′-disuccinimidyl tartrate (DST), N,N′-disuccinimidyl oxalate (DSO), suberic acid bis(N-hydroxysuccinimide ester), N,N′-disuccinimidyl glutarate (DSG), N,N′-disuccinimidyl suberate (DSS), N,N′-disuccinimidyl homobifunctional poly(ethylene glycol) (PEG) and combinations thereof; and a second hydrazidyl homo-bifunctional linker selected from the group consisting of adipic acid dihydrazide, succinic dihydrazide, oxalyl dihydrazide, 9,10-dihydro-9,10-ethanoanthracene-11,12-dicarboxylic dihydrazide, ethylmalonic acid dihydrazide, isophthalic acid dihydrazide, pimelic acid dihydrazide, carbohydrazide, thiocarbohydrazide, suberic acid dihydrazide, 6-hydrazinonicotinic hydrazide, 4-aminobenzoic hydrazide, 2-amino-isonicotinic acid hydrazide, and combinations thereof.

In some embodiments, the bifunctional linker includes a homo-bifunctional material containing succinimidyl, hydrazide, or aminooxyl functional groups; or a hetero-bifunctional material containing succinimidyl, hydrazide, or aminooxyl functional groups. In some embodiments, the homo-bifunctional linker includes N,N′-disuccinimidyl carbonate, adipic acid dihydrazide or any combination thereof.

In other embodiments, there is a first and a second bifunctional linker between the underlying scaffold and the glycan or functional element. In one embodiment, the first bifunctional linker is a disuccinimidyl linker, and the second bifunctional linker is a dihydrazide linker or a hydrazide-amine heterobifunctional linker. In this way a succinimidyl group of the first linker may attach to the second bifunctional linker through the hydrazide group (of the dihydrazide linker) or amine group (of the hydrazide amine linker). From there, the hydrazide functional group of the second linker is available to react with a glycan having a reducing sugar through its aldehyde or ketone groups. Thus, the underlying scaffold is attached through its amine terminal group to a succinimidyl group of the first linker. The second succinimidyl group of the first linker is then attached alternatively to a glycan through the glycan's amine group, and/or the second succinimidyl group of the first linker is attached through either an amine group or a hydrazide group of the second linker. The second functional group (hydrazide) of the second linker may then be used to attach to the aldehyde or ketone group of a glycan having a reducing end.

In particular embodiments, the linker(s) are formed of one or more functional groups selected from acrylamide, alkyne, amine, azide, biotin, carboxylic acid (COOH), dibenzocyclooctyne (DBCO), hydrazine, maleimide, phosphate, succinimidyl NHS ester and sulfhydryl mercapto (SH).

Additionally or alternatively, the linkers may include one or more affinity tag pairs. Affinity tag pairs are well known in the art and include epitope/antibody, biotin/avidin, biotin/streptavidin, biotin/neutravidin, glutathione-S-transferase/glutathione, maltose binding protein/amylase and maltose binding protein/maltose. Examples of suitable epitopes which may be used for epitope/antibody binding pairs include, but are not limited to, HA, FLAG, c-Myc, glutatione-S-transferase, His₆, GFP, DIG, biotin and avidin. Antibodies (both monoclonal and polyclonal and antigen-binding fragments thereof) which bind to these epitopes are well known in the art.

Affinity tags allow for highly flexible, modular assembly and disassembly of glycans and/or functional elements which are conjugated to affinity tags which form highly specific, noncovalent, physiochemical interactions with complementary affinity tags which are conjugated to the terminals of underlying scaffolds. The underlying scaffolds can be conjugated with a single species of affinity tag or with any combination of affinity tag species in any ratio. The ability to vary the number of species of affinity tags and their ratios conjugated to dendrimeric arms allows for exquisite control over the number and type of glycans and/or functional elements which may be attached to the scaffolds and their ratios.

In some embodiments, glycans and/or functional elements are tethered to the underlying scaffold using “click” chemistry. Click chemistry is the 1,3-dipolar cycloaddition of an azide and alkyne to form 1,2,3-triazole. While many other chemistries can used for coupling glycan to linker, the choice of click chemistry has several major advantages. The click chemistry functional groups (azide, alkyne, DBCO, etc.) are virtually non-existent in biological systems and other chemical groups therefore preventing unwanted reactions, including with glycans and proteins. The azidation of glycans appears very specific, e.g. on the reducing sugar only, and has a relatively high yield, >70% on most oligosaccharide tested (Tanaka, et al., Chem Commun (Camb), (23):3378-9, doi: 10.1039/b905761g (2009)). The azidation of the reducing sugar conserves chemical and structure properties, notably a close ring structure (Tanaka, et al., Chem Commun (Camb), (23):3378-9, doi: 10.1039/b905761g (2009)). This is not always the case with alternative tagging chemistries/methods. The click chemistry reaction is one of the most effective available for coupling two molecules. It is fast, reliable, specific, complete, accommodates many reaction conditions, and requires very minimal setup. With for example, DBCO+N3, only the two components are needed in solution to catalyze the reaction, nothing else. In the Examples Section below, the reducing end of glycans were derivatized with an azide (N3) tag, and click chemistry was used to couple the N3-derivatized glycans to an alkyne/DBCO-biotin 8-mer linker (7 arms alkyne/DBCO and 1 arm biotin).

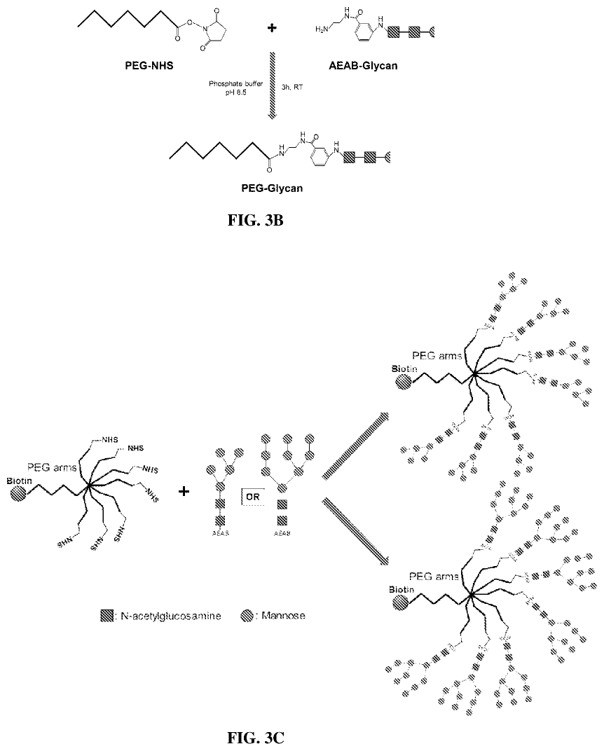

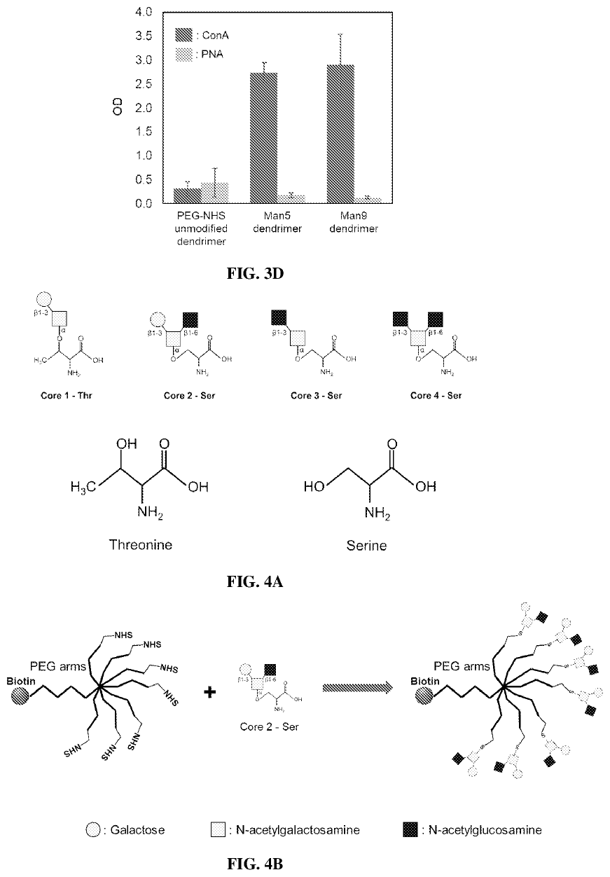

In some embodiments, glycans and/or functional elements are tethered to the underlying scaffold using the bifunctional fluorescent linker 2-amino-N-(2-aminoethyl)-benzamide (AEAB) (Song, et al., “Novel fluorescent glycan microarray strategy reveals ligands for galectins,” Chem Biol. 2009 Jan. 30; 16(1):36-47. doi: 10.1016/j.chembiol.2008.11.004. PMID: 19171304; PMCID: PMC2662446, which is specifically incorporated by reference herein in its entirety). AEAB can be conjugated through its arylamine group by reductive amination to free glycans to form glycan-AEABs (GAEABs), which can in turn be incorporated into the scaffold, e.g., via conjugation to one or more arms thereof. See, for example, the experiments described in Example 2 below and the Figures associated therewith, e.g., FIGS. 3A-3C. The glycan-AEAB conjugates (GAEABs) retain fluorescence similar to the glycan-2,6-diaminopyridine (DAP) conjugates, however, the arylamine is replaced with a primary alkylamine functional group for more efficient immobilization onto N-hydroxysuccinimide (NHS)-derivatized surfaces.

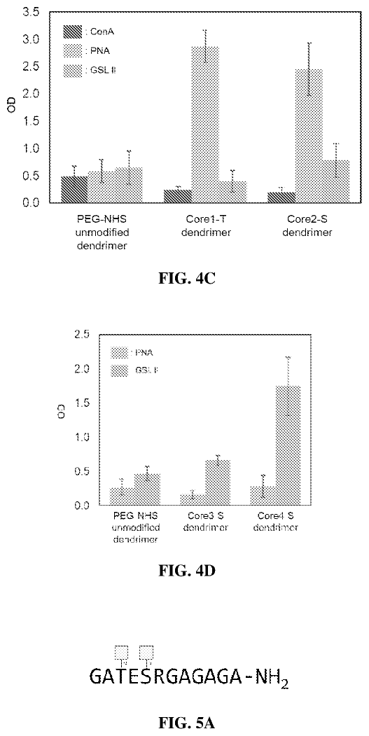

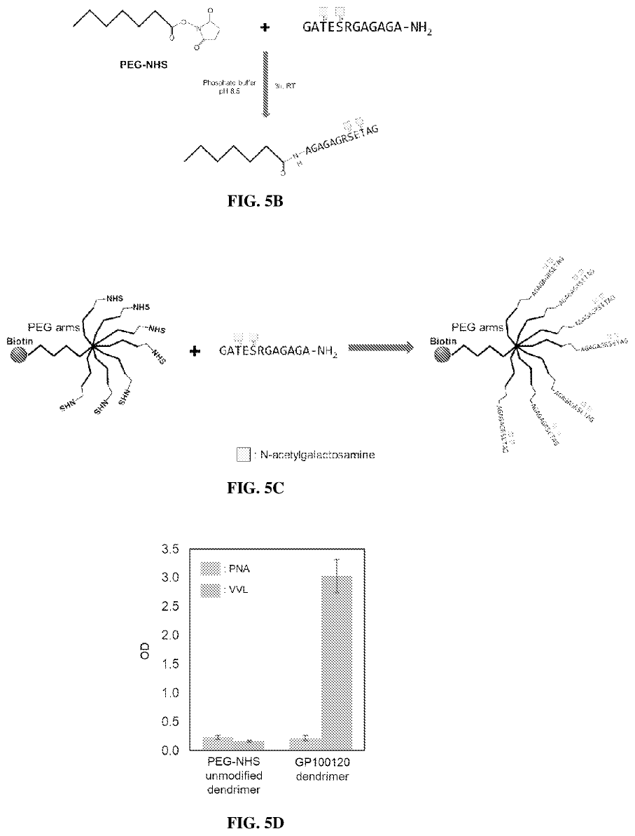

In some embodiments, an amino acid e.g., by itself or as part of a glycoamino acid or glycopeptide serves as a linker linking a glycan or glycan(s) to an arm of the scaffold. Glycopeptides can include one or more of the same or different glycans. Examples of dendrimers formed by glycans linked to single amino acids (e.g., serine and threonine) and a peptide (e.g., at serine and threonine residues) are provided in the experiments of Example 2 below, see, e.g., FIGS. 4A-4B and 5A-5C, respectively.

In some embodiments, the scaffold includes two or more different linkers (or bifunctional linkers) on one, two or more different terminal ends of the underlying scaffold. Thus, the scaffold can present two of more different terminal linkers to attachment of two or more different moieties (e.g., a glycan and another moiety). This approach can be used to more precisely control the ratio of glycans to other functional molecules. The scaffolds are typically multimeric glycan scaffolds, and thus typically present two or more glycans. In some embodiments, the scaffolds present 0, 1, 2, or more functional elements.

Linker/dendrimer configuration possibilities are substantial, and many are commercially available. By non-limiting example, commercial vendors offer virtually any custom synthesis possibilities of 8-arm PEG-X/Y with 1 arm with “Y” functional group and 7 arms with “X” functional group. Such scaffold can be used to prepare a multivalent glycan scaffold presenting 7 glycans and 1 functional element.

The foregoing are exemplary and non-limiting, and other configurations of linkers can be envisioned by one of skill in the art.

### 3. Glycans

The glycans presented by each scaffold can be any one or more different species of glycans. In some embodiments, the scaffold presents 2 or more copies of a single species of glycan (i.e., homogenous for the glycan). In other embodiments, the scaffold presents 1, 2, 3, or more copies of two or more different species of glycans (i.e., heterogenous for the glycans).

The glycans can be naturally occurring or synthetic glycans. The glycans can be obtained from commercial vendors or isolated from a biological source such as cells, tissues and glycoproteins.

Exemplary glycans include, but are not limited to, high-mannose N-glycans, complex N-glycans, hybrid N-glycans, O-GalNAc O-glycans, O-mannose O-glycans, O-GlcNAc O-glycans, blood groups and lewis antigen glycans, human milk oligosaccharides, glycosphingolipid glycans, glycosaminoglycans, bacterial glycans, and glycan-containing natural products. Examples of glycans which can be used also include all of the glycans specifically mentioned herein.

Exemplary commercially available glycans include, but are not limited to, all blood group glycans such as Blood group H type 2 trisaccharide, Lewis X, Sialyl Lewis A, 6-Sulfo Sialyl Lewis X, 3′- and 6′-sialylLacNAc, Globo-N-pentaose (Gb5), and Globo-N-tetraose (Gb4), etc.

Natural sources from which N-glycans can be enzymatically released include, but are not limited to, bovine fetuin, ribonuclease B, chicken ovalbumin, hen's egg yolk sialylglycopeptide, human serum IgGs and cultured cells including primary and immortalized cells (e.g., Jurkat, THP, Raji, Daudi) or mixtures of populations of any one or more of foregoing, including combinations of two or more different cell types. For each glycoprotein, the N-glycans from bovine fetuin, ribonuclease B, hen's egg yolk sialylglycopeptide, and chicken ovalbumin are rather limited in their heterogeneity. The N-glycans from bovine fetuin are mainly di- and tri-antennary N-glycans modified with 2, 3 or even 4 sialic acids (Jiang, et al., Anal Bioanal Chem., 409(2):421-9, doi: 10.1007/s00216-016-9690-x (2017)). The N-glycans from ribonuclease B are limited to the high-mannose type Man5-9 (Jiang, et al., Anal Bioanal Chem., 409(2):421-9, doi: 10.1007/s00216-016-9690-x (2017)). The N-glycans from hen's egg yolk sialylglycopeptide are exclusively composed of sialylated bi-antennary N-glycans (Seko et al., Biochim Biophys Acta., 1335(1-2):23-32, doi: 10.1016/s0304-4165(96)00118-3 (1997)). The N-glycans from chicken ovalbumin are almost exclusively composed of high-mannoses and a wide variety of N-acetylglucosamine-terminated N-glycans, with very little to no modifications with galactose, fucose, and sialic acid (Jiang, et al., Anal Bioanal Chem., 409(2):421-9, doi: 10.1007/s00216-016-9690-x (2017)).

O-glycans from mucins can be released by ammonium hydroxide and reduced with boric acid to then be suitable for azide modification with the protocol described in the examples below. Using commercially available mucins such as porcine stomach mucins (PSM) glycan dendrimers representing PSM O-glycans can be prepared.

Natural glycans can greatly vary in monosaccharide composition, size, and structure. Due to chemical/enzymatic synthesis and cost limitations commercial glycans are often restricted to structures consisting of 1-4 monosaccharide units and therefore only representing limited glycan epitopes. “Full” glycans also offer the possibility to present a certain glycan epitope in different context, e.g. Lewis X on an antenna of an N-glycan or on an O-glycan. Working with naturally derived glycans is representative of glycan actually occurring in nature. Further, natural glycans can be harvested in larger numbers. While preparation of synthetic glycans may be practically limited by cost and other resources, naturally derived glycans can be prepared in relatively large quantities. The disclosed compositions, devices, and methods encompass isolating and optionally utilizing up to hundreds of thousands of glycans.

Generally, glycans isolated from natural sources will be heterogeneous, particularly if isolated from a cell, tissue or organism. Heterogenous mixtures of glycans can be separated e.g., by HPLC, in order to prepare homogeneous, i.e., monospecific but multivalent, glycan dendrimers. It is also possible to generate glycan dendrimers with glycan closely related, only different by one monosaccharide or one linkage, thus creating a library that can be used for fine screening of potential glycan ligands.

As introduced above, in some embodiments, a single scaffold presents two or more glycans in combination. The glycans can be presented in any conceivable ratio based on the number of available arms and the number of glycan species being linked. As an illustrative, non-limiting example, two glycans, G1 and G2, alone or in combination with a functional element, FE, are coupled to an 8-arm dendrimer (e.g., 8-arm PEG) at a ratio of 1:6:1, 2:5:1, 3:4:1, 4:3:1, 5:2:1, 6:1:1, 1:7:0, 2:6:0, 3:5:0, 4:4:0, 5:3:0, 6:2:0, 7:1:0 of G1:G2:FE. Such dendrimers can be used experimentally to assay/compare agonist/antagonist binding effect of 2 different glycans, e.g., u2-3 vs u2-6 linkage of sialic acids.

Methods of isolating glycans and preparing them for linkage to the underlying scaffold are discussed in the Examples below, references cited therein, and are otherwise known in the art based on the reactive group that will be used to control the linking. See, e.g., Song, et al., “Oxidative release of natural glycans for functional glycomics,” Nat Methods 13, 528-534 (2016), doi.org/10.1038/nmeth.3861. For example, hydrazinolysis has been used for both N- and O-glycan analysis and preparation but it is limited due to the reagent toxicity and glycan structural integrity. Ammonium salt treatment was originally developed for O-glycans and found to also be effective for N-glycans. Pronase digestion of glycoproteins can release glycoamino acids, which can be directly functionalized or further trimmed with of N-bromosuccinimide (NBS) for further functionalization. One-pot (3-elimination and tagging with 1-phenyl-3-methyl-5-pyrazolone (PMP) have shown to be effective analytical methods for O-glycan release. Endoceramidases are available to release the lipid moiety from glycosphingolipids (GSLs) for glycan analysis and ozone initiated glycan release from glycosphingolipids under basic or neutral conditions can be potentially used for large scale preparation.

Tanaka et al., initially described and established the glycan azidation process utilized in the Examples below (Tanaka, et al., Chem Commun (Camb), (23):3378-9, doi: 10.1039/b905761g (2009)), and see also, Streichert et al., for both glycan azidation and click chemistry coupling (Streichert, et al., Chembiochem., 20(15):1914-8, doi: 10.1002/cbic.201900023 (2019)), each of which are specifically incorporated by reference herein in their entireties). Tanaka showed a reaction by which the terminal reducing end of any glycan could be derivatized with an azide (N3) tag by selective activation of the anomeric hydroxy group while preserving other functional groups of the modified monosaccharide residue, including the N-acetyl groups of the non-reducing GlcNAc of N-glycans. Copper(I)-catalyzed azide-alkyne cycloaddition reaction, or “click chemistry”, was introduced two decades ago by Rostovtsev et al., (Rostovtsev, et al., Angew Chem Int Ed Engl., 41(14):2596-9, doi: 10.1002/1521-3773(20020715)41:14<2596::AID-ANIE2596>3.0.CO;2-4 (2002)) and was adapted for all sorts of biomolecules including glycans (Baskin, et al., Proc Natl Acad Sci USA, 104(43):16793-7, doi: 10.1073/pnas.0707090104 (2007); Zhang, et al., Molecules, 18(6):7145-59. doi: 10.3390/molecules18067145 2013; Streichert, et al., Chembiochem., 20(15):1914-8, doi: 10.1002/cbic.201900023 (2019)).

N3-tagged glycans can also be clicked onto alkyne or DBCO magnetic beads. Such magnetic beads can be used for pull-down assays and/or flow cytometry type experiments. Specific examples include blood group H type 2 trisaccharide, human serum IgG-derived N-glycans, bovine fetuin-derived N-glycans and chicken ovalbumin-derived N-glycans.

Song, et al., described a method of release glycans from diverse types of biological samples. The oxidative strategy involves using bleach, and is effective to release all types of free reducing N-glycans and O-glycan-acids from glycoproteins, and glycan nitriles from glycosphingolipids. Released glycans are directly useful in glycomic analyses and/or can be derivatized, e.g., fluorescently. Briefly, NaClO, which is known to degrade proteins, effectively and selectively degrades the aglycon portion of native glycoconjugates to release intact glycans. Brief treatment of glycoproteins with NaClO releases free N-glycans, which can be specifically derivatized through the reducing end. NaClO degrades glycoproteins in minutes, and released glycans are easily visualized by their high mobility compared to untreated glycoproteins in thin-layer chromatography (TLC). The process is applicable to all types of common glycoproteins, and has been demonstrated using, for example, ovalbumin, bovine IgG and horseradish peroxidase (HRP), the last of which is resistant to PNGase F digestion owing to core 3-fucose modification of N-glycans. The typically labile sialic acid residues also are well preserved, as demonstrated with bovine fetuin. N-glycans derived in this fashion react with amine-based tags, such as the bifunctional fluorescent linker 2-amino-N-(2-aminoethyl)benzamide (AEAB) and other fluorescent tags and linkers (e.g., as discussed above), to facilitate chromatographic separation and purification and/or incorporation into the scaffold. See, e.g., Song, et al., “Oxidative release of natural glycans for functional glycomics,” Nat Methods 13, 528-534 (2016), //doi.org/10.1038/nmeth.3861, Song, et al., “Novel fluorescent glycan microarray strategy reveals ligands for galectins,” Chem Biol. 2009 Jan. 30; 16(1):36-47. doi: 10.1016/j.chembiol.2008.11.004. PMID: 19171304; PMCID: PMC2662446, U.S. Patent Application No. 2013/0331280, and U.S. Patent Application No. 2020/0262940, each of which is specifically incorporated by reference herein in its entirety.

Previous attempts to generate “glyco-structures” has focused on building “more multivalent” structures with 1-3 monosaccharides attached to it (Bernardi et al., Chem Soc Rev., 42(11):4709-27, doi: 10.1039/c2cs35408j (2013), Mousavifar, et al., Molecules., 26(9), doi: 10.3390/molecules26092428 (2021), Reichardt, et al., Chem Soc Rev., 42(10):4358-76, doi: 10.1039/c2cs35427f (2013), Spinelli, et al., Chem Soc Rev., 42(11):4557-73, doi: 10.1039/c2cs35406c (2013)). Only a very limited number of studies tried to develop a methodology to a more complex set of glycans (commercial or naturally released) (Smirnov, et al. Small., 16(46):e2004831, doi: 10.1002/smll.202004831 (2020)). In some embodiments, the disclosed scaffolds feature not just simple multimerization of glycan presentation but present larger set of glycans (i.e., 2, 3, 4, 5, 6, 7, 8, 9, 10, or more different species of glycans on the same scaffold and/or multiple scaffolds with different species of glycans linked thereto), including, especially, naturally released glycans.

In other embodiments, an azide group or hydroxy group is present on or integrated onto an amino acid e.g., by itself or as part of a glycoamino acid or glycopeptide, e.g., a synthetic glycoamino acid or glycopeptide to generate glycoamino acid or glycopeptide dendrimers (i.e., rather than glycan (only) dendrimers). Glycopeptides can include one or more of the same or different glycans. Examples of dendrimers formed by glycans linked to single amino acids (e.g., serine and threonine) and a peptide (e.g., at serine and threonine residues) are provided in the experiments of Example 2 below, see, e.g., FIGS. 4A-4B and 5A-5C, respectively. In such cases, the amino acid and/or peptide can be viewed as a linker between the glycan and arms of the dendrimer.

Such embodiments can be used to explore glycopeptide interactions. For example, glycopeptides known to be required for interaction (meaning both glycan and peptide parts are needed) can be investigated. More rare glycosylation types such as O-Fuc, O-Man, O-GlcNAc (extracellular) are specifically found on a few defined peptide sequences and usually only a few monosaccharide long (which can be more easily generated synthetically). These would be ideal glycopeptides to be used on glycopeptide dendrimer. For example, nuclear proteins modified by GlcNAc may be useful for pulling out interacting proteins.

### 4. Functional Groups

One or more of the scaffold's arms can be functionalized with a functional element. Functional elements can be linked to the scaffold using linkers as discussed above. The moieties are typically selected based on the planned use of the scaffold. The functional element can be, for example, a small molecule, protein or polypeptide, carbohydrate, nucleic acid or a combination thereof. The functional elements can serve a variety of different functions; such as, tracking or otherwise monitoring or identifying the scaffold, characterizing the binding activity of the scaffold, and isolating the scaffold. The scaffold can include a combination of two or more of the same or different types of moieties. The moieties can be in series or in parallel. In other words, two or more functional elements can be tethered to the same arm of the underlying scaffold (i.e., in series) as illustrated in FIG. 1B, or to separate arms of the underlying scaffold (i.e., in parallel). Exemplary functional elements are discussed in more detail below, and include, but are not limited to oligomeric bioconjugates (Manimala, et al., Glycobiology., 17(8):17C-23C, doi: 10.1093/glycob/cwm047 (2007)), polymers (Becer, Macromol Rapid Commun., 33(9):742-52, doi: 10.1002/marc.201200055 (2012)), quantum dots ( )Hild, et al., Eur J Pharm Biopharm., 2008; 68(2):153-68, doi: 10.1016/j.ejpb.2007.06.009 (2008), and nanoparticles (Song, et al., J Am Chem Soc., 132(33):11428-30, doi: 10.1021/ja103351m (2010), Wang, et al., Adv Sci (Weinh), 7(16):2001264. Epub 2020/08/25. doi: 10.1002/advs.202001264 (2020)).

### a. Imaging and Contrast Agents

In some embodiments, the scaffold is functionalized with an agent for identifying the presence, determining the location, and/or quantitating the amount of scaffold. Agents useful for this purpose include fluorescent tags, radionuclides and contrast agents.

Suitable imaging agents include, but are not limited to, fluorescent molecules such as those described by Molecular Probes (Handbook of fluorescent probes and research products), such as Rhodamine, fluorescein, Texas red, Acridine Orange, Alexa Fluor (various), Allophycocyanin, 7-aminoactinomycin D, BOBO-1, BODIPY (various), Calcein, Calcium Crimson, Calcium green, Calcium Orange, 6-carboxyrhodamine 6G, Cascade blue, Cascade yellow, DAPI, DiA, DiD, Dil, DiO, DiR, ELF 97, Eosin, ER Tracker Blue-White, EthD-1, Ethidium bromide, Fluo-3, Fluo4, FM1-43, FM4-64, Fura-2, Fura Red, Hoechst 33258, Hoechst 33342, 7-hydroxy-4-methylcoumarin, Indo-1, JC-1, JC-9, JOE dye, Lissamine rhodamine B, Lucifer Yellow CH, LysoSensor Blue DND-167, LysoSensor Green, LysoSensor Yellow/Blu, Lysotracker Green FM, Magnesium Green, Marina Blue, Mitotracker Green FM, Mitotracker Orange CMTMRos, MitoTracker Red CMXRos, Monobromobimane, NBD amines, NeruoTrace 500/525 green, Nile red, Oregon Green, Pacific Blue. POP-1, Propidium iodide, Rhodamine 110, Rhodamine Red, R-Phycoerythrin, Resorfin, RH414, Rhod-2, Rhodamine Green, Rhodamine 123, ROX dye, Sodium Green, SYTO blue (various), SYTO green (Various), SYTO orange (various), SYTOX blue, SYTOX green, SYTOX orange, Tetramethylrhodamine B, TOT-1, TOT-3, X-rhod-1, YOYO-1, YOYO-3, and 2-amino-N-(2-aminoethyl)benzamide (AEAB).

NanoLuc tags can be added to a peptide sequence and can be used for, luciferase imaging and or to label a dendrimer, e.g. in place of biotin.

Additionally, radionuclides can be used as imaging agents. Suitable radionuclides include, but are not limited to radioactive species of Fe(III), Fe(II), Cu(II), Mg(II), Ca(II), and Zn(I1) Indium, Gallium and Technetium. Other suitable contrast agents include metal ions generally used for chelation in paramagnetic T1-type MIR contrast agents, and include di- and tri-valent cations such as copper, chromium, iron, gadolinium, manganese, erbium, europium, dysprosium and holmium. Metal ions that can be chelated and used for radionuclide imaging, include, but are not limited to metals such as gallium, germanium, cobalt, calcium, indium, iridium, rubidium, yttrium, ruthenium, yttrium, technetium, rhenium, platinum, thallium and samarium. Additionally metal ions known to be useful in neutron-capture radiation therapy include boron and other metals with large nuclear cross-sections. Also suitable are metal ions useful in ultrasound contrast, and X-ray contrast compositions.

### b. Antibodies and Ligands

In some embodiments, the scaffold is functionalized with a nucleic acid or peptide or proteins such as an antibody or receptor ligand. Ligands may be, for example, growth factors, cytokines and chemokines, including the chemokines. Antibodies can be polyclonal, monoclonal, fragments, recombinant, or single chain, many of which are commercially available or readily obtained using standard techniques. The antibodies can be a fragment of a full-length antibody, or a fusion protein formed for segments of the antibody. Exemplary antibody fragments and fusions include, but are not limited to, single chain antibodies, single chain variable fragments (scFv), di-scFv, tri-scFv, diabody, triabody, teratbody, disulfide-linked Fvs (sdFv), Fab′, F(ab′)₂, Fv, and single domain antibody fragments (sdAb).

Ligands and antibodies can be used to enhance the targeting or attachment of the scaffolds to surfaces or cells, isolation of scaffold (i.e., affinity chromatography), etc.

### c. Tags for Isolation

In some embodiments, the scaffold is functionalized with a tag to enhance isolation or purification of the scaffold. As introduced above, this can be an antibody or ligand, or polypeptide purification tag. Polypeptide purification tags are known in the art and include, but are not limited to, His tags which typically include six or more, typically consecutive, histidine residues; green fluorescent protein (GFP), glutathione S-transferase (GST), polyhistidine, c-myc, hemagglutinin, Flag™ tag (Kodak, New Haven, CT), maltose E binding protein, protein A, and a small ubiquitin-related modifier (SUMO). More specific examples include FLAG tags including the sequence DYKDDDDK (SEQ ID NO:1); haemagglutinin (HA) tags including the sequence YPYDVP (SEQ ID NO:2); or MYC tags including the sequence ILKKATAYIL (SEQ ID NO:3) or EQKLISEEDL (SEQ ID NO:4). Methods of using purification tags to facilitate protein purification are known in the art and include, for example, a chromatography step wherein the tag reversibly binds to a chromatography resin. Similar methods can be used to isolate scaffolds.

### d. Barcodes

In some embodiments, the underlying scaffold and/or the glycan is functionalized with a nucleic acid barcode, such that ultimately the glycan scaffold is functionalized with one or more nucleic acid bar codes. For example, in some embodiments, a barcode is attached to the glycan ligand and the glycan-barcode compound then attached to the underlying scaffold to form the multimeric structure. In other embodiments, the barcode and glycan are each separately attached the underlying scaffold. A nucleic acid barcode, also referred to as a barcode, unique molecular identifier, or UMI, is a short sequence of nucleotides (for example, DNA, RNA, or combinations thereof) that is used as an identifier for an associated molecule, such as a target molecule and/or target nucleic acid, for example cell type or phenotype, a particular genomic perturbagens, or as used herein, more typically a multivalent glycan scaffold.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. One or more nucleic acid barcodes and/or UMIs can be attached, or “tagged,” on one or more arms of the glycan scaffold. This attachment can be direct (for example, covalent or noncovalent binding of the barcode to the target molecule) or indirect (for example, via an additional molecule, for example, a specific binding agent, such as an antibody (or other protein) or a barcode receiving adaptor (or other nucleic acid molecule). In some embodiments, the barcode is in series with another functional element such as a fluorescent tag, purification tag, etc., as illustrated in FIG. 1B. The UMI can be flanked a common amplification sequence, that allow two or more different UMI sequences to amplified using common primers. Thus, the barcode can consist of a UMI sequence, to include a UMI sequence and one or more additional sequences such as a common primer binding sequence.

The glycan scaffold can be labeled with multiple nucleic acid barcodes in combinatorial fashion, such as a nucleic acid barcode concatemer. Typically, a nucleic acid barcode is used to identify a glycan scaffold as being from a particular compartment or library, having a particular physical property (for example, glycan(s), other functional elements, etc.), or having been subject to certain treatment conditions, contacted with certain cell types, etc. The glycan scaffold can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example glycan(s), other functional elements, etc.) nucleic acid barcodes.

To identify the UMI, the nucleic acid is optionally amplified (e.g., by PCR), and the UMI is “read” by, for example, sequencing, hybridization to a known complementary sequence (e.g., microarray, fluorescently-tagged nucleic acid, etc.), or any other suitable means.

### e. Active Agents

In some embodiments, the multivalent glycan scaffolds are functionalized one or more active agents that can be delivered to, e.g., a subject or cells, using the disclosed scaffolds.

Active agents to be delivered include therapeutic, nutritional, diagnostic, and prophylactic agents. The active agents can be small molecule active agents or biomacromolecules, such as proteins, polypeptides, or nucleic acids. Suitable small molecule active agents include organic and organometallic compounds. The small molecule active agents can be a hydrophilic, hydrophobic, or amphiphilic compound.

Exemplary therapeutic agents that can be incorporated onto scaffolds include antigens, e.g., tumor antigens, CD4+ T-cell epitopes, cytokines, chemotherapeutic agents, radionuclides, small molecule signal transduction inhibitors, photothermal antennas, monoclonal antibodies, immunologic danger signaling molecules, other immunotherapeutics, enzymes, antibiotics, antivirals (especially protease inhibitors alone or in combination with nucleosides for treatment of HIV or Hepatitis B or C), anti-parasites (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, immunomodulators (including ligands that bind to Toll-Like Receptors (including but not limited to CpG oligonucleotides) to activate the innate immune system, molecules that mobilize and optimize the adaptive immune system, molecules that activate or up-regulate the action of cytotoxic T lymphocytes, natural killer cells and helper T-cells, and molecules that deactivate or down-regulate suppressor or regulatory T-cells), agents that promote uptake of multivalent glycan scaffolds into cells (including dendritic cells and other antigen-presenting cells), nutraceuticals such as vitamins, and oligonucleotide drugs (including DNA, RNAs, antisense, aptamers, small interfering RNAs, ribozymes, external guide sequences for ribonuclease P, triplex forming agents, crispr guide RNA (e.g., gRNA, sgRNA), etc.).

In a particular embodiment, the therapeutic agent is a bivalent structure containing an E3 ligase ligand and a binding agent or ligand for the protein to be destroyed (e.g., PROTACS®). Rather than acting as a conventional enzyme inhibitor, such agents work by inducing selective intracellular proteolysis (i.e., ubiquitination of the target and destruction (proteolysis) by the proteosome). PROTACS® require intracellular uptake, which is likely to happen when multimeric glycan ligands crosslink cellular GBPs, and cell type specificity, which can be achieved with the glycan ligand:GBP interaction. For review see Scheepstra, et al., Comput Struct Biotechnol J, 17:160-176. doi: 10.1016/j.csbj.2019.01.006 (2019)).

Exemplary diagnostic agents are introduced above and include, but are not limited to, paramagnetic molecules, fluorescent compounds, magnetic molecules, and radionuclides, x-ray imaging agents, and contrast agents.

Toxins and “killing agents” could be harnessed to the glycan dendrimers to target and kill cancer cells/tumors (Cousin, et al., Int J Mol Sci., 17(9), doi: 10.3390/ijms17091566 (2016), Torres-Perez, et al., Front Oncol., 10:605037, doi: 10.3389/fonc.2020.605037 (2020), Veprek, et al., J Med Chem., 49(21):6400-7, doi: 10.1021/jm050741g (2006), Smirnov, et al. Small., 16(46):e2004831, doi: 10.1002/smll.202004831 (2020)). Thus, in some embodiments, the active agent is an anti-cancer agent. In certain embodiments, the multivalent glycan scaffold includes one or more anti-cancer agents. Representative anti-cancer agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (Nexavar®), erlotinib (Tarceva®), pazopanib, axitinib, and lapatinib; transforming growth factor-α or transforming growth factor-β inhibitors, and antibodies to the epidermal growth factor receptor such as panitumumab (VECTIBIX®) and cetuximab (ERBITUX®).

In certain embodiments, the multivalent glycan scaffold includes one or more immunomodulatory agents. Exemplary immunomodulatory agents include cytokines, xanthines, interleukins, interferons, oligodeoxynucleotides, glucans, growth factors (e.g., TNF, CSF, GM-CSF and G-CSF), hormones such as estrogens (diethylstilbestrol, estradiol), androgens (testosterone, HALOTESTIN® (fluoxymesterone)), progestins (MEGACE® (megestrol acetate), PROVERA® (medroxyprogesterone acetate)), and corticosteroids (prednisone, dexamethasone, hydrocortisone).

Examples of immunological adjuvants that can be associated with the particles include, but are not limited to, TLR ligands, C-Type Lectin Receptor ligands, NOD-Like Receptor ligands, RLR ligands, and RAGE ligands. TLR ligands can include lipopolysaccharide (LPS) and derivatives thereof, as well as lipid A and derivatives there of including, but not limited to, monophosphoryl lipid A (MPL), glycopyranosyl lipid A, PET-lipid A, and 3-O-desacyl-4′-monophosphoryl lipid A. In a specific embodiment, the immunological adjuvant is MPL. In another embodiment, the immunological adjuvant is LPS. TLR ligands can also include, but are not limited to, TLR3 ligands (e.g., polyinosinic-polycytidylic acid (poly(I:C)), TLR7 ligands (e.g., imiquimod and resiquimod), and TLR9 ligands.

The multivalent glycan scaffold may also include antigens and/or adjuvants (i.e., molecules enhancing an immune response). Antigens can be peptides, proteins, polysaccharides, saccharides, lipids, glycolipids, nucleic acids, or combinations thereof. The antigen can be derived from any source, including, but not limited to, a virus, bacterium, parasite, plant, protozoan, fungus, tissue or transformed cell such as a cancer or leukemic cell and can be a whole cell or immunogenic component thereof, e.g., cell wall components or molecular components thereof.

Suitable antigens are known in the art and are available from commercial government and scientific sources. The antigens may be purified or partially purified polypeptides derived from tumors or viral or bacterial sources. Criteria for identifying and selecting effective antigenic peptides (e.g., minimal peptide sequences capable of eliciting an immune response) can be found in the art. For example, A postolopoulos, et al. (Curr. Opin. Mol. Ther., 2:29-36 (2000)), discusses the strategy for identifying minimal antigenic peptide sequences based on an understanding of the three-dimensional structure of an antigen-presenting molecule and its interaction with both an antigenic peptide and T-cell receptor. Shastri, (Curr. Opin. Immunol., 8:271-7 (1996)), disclose how to distinguish rare peptides that serve to activate T cells from the thousands peptides normally bound to MHC molecules. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids. Peptide, protein, and DNA based vaccines may be used to induce immunity to various diseases or conditions. Cell-mediated immunity is needed to detect and destroy virus-infected cells. Most traditional vaccines (e.g. protein-based vaccines) can only induce humoral immunity. Nucleic acid vaccines, such as RNA and DNA-based vaccines, represents a unique means to vaccinate against a virus or parasite because nucleic acid based vaccines can induce both humoral and cell-mediated immunity. In addition, nucleic acid based vaccines are potentially safer than traditional vaccines, and can be more stable and more cost-effective for manufacturing and storage. Nucleic acid vaccines include two major components, carriers (or delivery vehicles) and nucleic acids encoding antigens. Carriers protect the nucleic acids from degradation, and can facilitate their entry to specific tissues or cells and expression at an efficient level.

In some embodiments, the antigen is a tolerogenic antigen. As used here, “tolerogenic” means the condition or capability of stimulating or increasing tolerance. A suitable tolerogenic antigen is selected based on the desired therapeutic outcome and the disease, disorder, or condition being treated. Exemplary antigens are known in the art. See, for example, U.S. Published Application No. 2014/0356384, which is specifically incorporated by reference herein in its entirety, discusses that tolerogenic antigens can be derived from a therapeutic agent protein to which tolerance is desired, from proteins that are administered to humans that are deficient in the protein, therapeutic antibodies and antibody-like molecules, proteins that are nonhuman, human allograft transplantation antigens, self-antigens against which a patient has developed an autoimmune response or may develop an autoimmune response, a foreign antigen against which a patient has developed an unwanted immune response (e.g., food antigens or drug antigens). It is also possible to induce tolerance by administering an antigen in the context of a tolerance inducing signal. CD22 is an “inhibitory” receptor on B lymphocytes that recognizes a glycan ligand (e.g., Neu5Acα1-6 Galμ1-4GlcNAc, and/or Neu5Acu2-6Galβ1-4(6S)GlcNAc. When an antigen is bound by the B cell antigen receptor (BCR) and CD22 is also bound by its ligand, the B cell becomes non-responsive or anergic. Qadura, et al., Haemophilia, 17:288e95 (2017), Orgel, et al., J Allergy Clin Immunol 2017; 139:366e9. e2, and Bednar, et al., ACS Chem Biol, 14:644e54 (2019).

In certain embodiments, the multivalent glycan scaffold core contains two or more active agents.

### f. Particles

In some embodiments, the scaffold functionalizes a delivery vehicle such as a nanoparticle, microparticle, micelle, synthetic lipoprotein particle, or carbon nanotube. Such delivery vehicles can be loaded with, and used to facilitate delivery of, other materials such as active agents, including those specifically mentioned above. In some embodiments, the delivery vehicles provide controlled release of the compound. In some embodiments, release of the active agent is controlled by diffusion of the compound out of the particles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof.

Functional elements can be linked to scaffolds using the same or similar means as discussed above (e.g., linkers and glycans), or using other methods that are known in the art. In Sago et al., RNA was encapsulated in lipid nanoparticles via phase mixing, no particular chemistry (Sago, et al., Proc Nat Acad Sci USA., 115(42):E9944-E52, doi: 10.1073/pnas.1811276115 (2018)). Sojitra et al., used a glycan-phage conjugates strategy where each phage incorporates a distinct DNA encoded barcodes. Desired glycans are chemically conjugated (via click chemistry) to a subset of the 2,700 copies of the major coat protein pVIII to produce multivalent display. IDT commercializes functionalized DNA, dithiol modified for example. These can be coupled to the glycan scaffold with a maleimide thiol-reactive group. DBCO-conjugated DNA from IDT has also been used to couple DNA to carboxylated polystyrene microsphere via an amine-PEG-N3 linker (Oh, et al., Langmuir., 36(13):3583-9, doi: 10.1021/acs.langmuir.9b03386 (2020)).

### B. Compositions Including Multivalent Glycan Scaffolds

Compositions including the disclosed multivalent glycan scaffolds are provided. The compositions can be, for example, a surface or substrate to which the scaffold is covalently or non-covalently linked to, or a solution within which the scaffold is disbursed. In some embodiments, the surface, substrate, solution, etc., include two or more scaffolds which may be the same species of scaffold (e.g., with the same underlying scaffold, glycan(s) and optional functional elements), different species of scaffold (e.g., with different underlying scaffold, glycan(s), and/or functional elements), or a combination thereof.

For example, in some embodiments, the surface, substrate, solution, etc. includes at least 1, 5, 10, 25, 50, 75,100,150, 250, 500, 1,000, 1,500, 2,500, 5,000, 10,000 of one or more multivalent glycan scaffolds of the same species or different species.

A collection of two or more different species of scaffolds can be referred to as a library. In some embodiments, the surface, substrate, solution, etc. includes a library of 5, 10, 25, 50, 75,100,150, 250, 500, 1,000, 1,500, 2,500, 5,000, 10,000 or more species of different multivalent glycan scaffolds.

### 1. Surfaces and Substrates

The disclosed multivalent glycan scaffolds can be covalently or non-covalently linked to a variety of surfaces and substrates. Substrate can include, but are not limited to, transparent and refractive solid-phase material, such as glass (such as silicate glass), ceramic, indium tin oxide (ITO), metal, plastic polymer and silica slides. Plastic polymers include such as polystyrene (Example 8), polypropylene, polycarbonate, polyethylene, high-density polyethylene (HDPE), polyvinyl chloride, polyamide, acrylonitrile butadiene styrene (ABS) and Polyurethane. The surfaces and substrates include but not limited to, beads, dishes including tissue culture dishes, plates including microtiter plates, microscope slides, point-of-care diagnostic chips, miniaturized biosensors, micro-reactors, and microelectrodes. See also U.S. Published Application No. 2019/0128881 which is specifically incorporated by reference herein in its entirety.

In some embodiments the multivalent glycan scaffold is attached to a surfaces or substrate through one or more of its arms. More particularly, in some embodiments, a linker or bifunctional linker links the scaffold to the surface or substrate. Any of the linkers or bifunctional linkers discussed herein or elsewhere can be used.

In some embodiments, the surface is a chromatography column. In such embodiments, a multivalent glycan scaffold having one of a pair of affinity tags (e.g., His tag) is bound to the column surface having the corresponding/second of the pair of affinity tags (e.g., nickel, cobalt, or copper ions in/on the column).

### 2. Cells and Proteins

As discussed in more detail below, the disclosed multivalent glycan scaffolds can be used in various cell and protein binding assays. Such assays can be used to identify glycan-to-cell and/or glycan-to-protein/receptor binding events and activity, and to isolate cells or proteins based thereon. Thus, in some embodiments, the multivalent glycan scaffolds are bound to cells or tissue or organs and/or proteins, to form a multivalent glycan scaffold-cell and/or multivalent glycan scaffold-protein complex.

In preferred embodiments, the binding is modulated through the glycans, optionally but preferably by binding to a glycan binding molecule such as a glycan binding protein. The binding is typically non-covalent, and can occur in the context of cell surface, or in another context, such as free or immobilized protein.

The protein can be recombinant or naturally occurring, and can be purified or unpurified (e.g., in a lysate). The protein can be a cell surface protein, e.g., on the surface of a cell, or a soluble protein.

The binding can be transient or stable.

The cells can be, for example, primary cells or established cell lines. Exemplary cells, tissues, and organs, include, but are not limited to, embryonic cells, immune cells, stem cells including unipotent cells, multipotent cells, and pluripotent cells; embryonic stem cells, and adult stem cells such as hematopoietic stem cells, mesenchymal stem cells, epithelial stem cells, neuronal stem cells, and muscle satellite cells, as well as differentiated cells including, but not limited to, cells derived from ectoderm, endoderm, and mesoderm, including but not limited to, fibroblasts, parenchymal cells, hematopoietic cells, white blood cells including lymphocytes, platelets, stromal cells, fat cells, lung cells, ductal cells, bone cells including osteoclasts, epithelial cells and tissue including skin cells such as keratinocytes, muscle tissue including smooth muscle, skeletal muscle, and cardiac muscle, vascular tissue including endothelial cells, liver tissue including hepatocytes, and nervous tissue including neurons, glia such as astroglia or astrocytes, oligoglia or oligodendrocytes, microglia, beta cells, pituitary cells, synovial lining cells, ovarian cells, testicular cells, fibroblasts, B cells, T cells, reticulocytes, leukocytes, granulocytes, tumor cells, or any combination thereof. As used herein, tumor cells include primary cancer cells as well as metastatic cells. Tumor cell is sometimes used herein interchangeably with cancer cell but also encompasses non-malignant (non-cancerous) cells exhibiting increased proliferation as compared to a normal cell.

Cells may be isolated from a subject. The subject can be a mammalian subject. Exemplary subjects include, but are not limited to humans, monkeys, dogs, cats, mice, rats, cows, horses, camels, goats and sheep. In some embodiments, the subject is a human. In some embodiments, the subject has or is suspected of having a tumor, e.g., a malignant tumor.

The cells can be obtained from any part of the subject, for example, from one or more organs or tissues of the subject. For example, and not to be limiting, the one or more cells can be from one or more tissues or organs selected from the group consisting of heart, liver, lung, muscle, testes, blood, brain, spleen, breast, skin, pancreas, mouth, esophagus, stomach, small intestine, large intestine, gallbladder and kidney of the subject. The one or more cells can be obtained from cancerous tissue, e.g., a tumor, or from non-cancerous tissue. The one or more cells can be obtained from one or more cells from one or more organs or tissues of a healthy subject or a subject with a particular disease or disorder. In some embodiments, the one or more cells can be obtained from one or more organs or tissues of a subject with cancer. Optionally, the one or more cells can be obtained from a biopsy of a tissue or organ of the subject.

In some embodiments, a population of cells, i.e., two or more cells, is obtained from a tissue or organ of the subject. Optionally, one or more populations of cells can be obtained from one or more tissues or organs of the subject. Optionally, a subpopulation of one or more cells can be isolated from a population of cells obtained from a tissue or organ of the subject, for example, by fluorescence activated cell sorting (FACS), magnetic-activated cell sorting (MACS), ELISA or microfluidic based sorting to isolate one or more specific cell types from the population of cells. By isolating specific cell types from a population of cells, the methods provided herein can be used to identify nanoparticles suitable for delivery to one or more specific cell types, or subpopulations of cells, in a tissue or organ of the subject. For example, tumor cells can be isolated using one or more cancer markers from a sample containing a population of liver cells comprising cancerous and non-cancerous cells.

Cellular compositions are also provided are refer to composition having at least one cell. In some embodiments, the cellular composition includes a single cell. In some embodiments, the cellular composition includes at least two, three, four, five, 10, 25, 50,100,500, 1,000 or more cells. Cellular compositions can be obtained from various different sources, such as in vivo sources, such as a bodily fluid or tissue sample from an animal, or in vitro sources, such as from cultured cells or cultured tissues. Bodily fluid and tissue samples often contain multiple cell types, for example brain tissue may comprise neurons, glial cells, and many other types of cells. In a particular embodiment, the cells are peripheral blood mononuclear cells (PBMC) from blood of an animal. Accordingly, in some embodiments, the cellular composition includes at least two, three, four, or five cell types. Cellular compositions may also include media, such as cell culture media.

Any one or more cells or cellular compositions mentioned here can be used in any of the methods discussed below.

### 3. Media and Pharmaceutical Compositions

In some embodiments, the multivalent glycan scaffolds alone, or covalently or non-covalently attached to a surface, substrate, cell, protein, etc., is bathed in a solution, preferably a media or pharmaceutically acceptable carrier.

The solution can be obtained from various different sources, such as in vivo sources, such as a bodily fluid or synthetic sources such as a pharmaceutical composition or media, such as cell culture media. The media may vary depending upon the sample and cell type used. In some embodiments, the cellular composition comprises one or more of: amino acids, vitamins, inorganic salts, glucose, serum, growth factors, hormones, and attachment factors. Exemplary media include Ames' Medium, BGJb Medium with or without the Fitton-Jackson Modification, Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, William's Medium E, Dulbecco's Modified Eagle Medium (DMEM) and Eagle's Minimum Essential Medium (EMEM).

Pharmaceutical compositions can be suitable for, but need not actually be, administered to a subject e.g., by parenteral (intramuscular (IM), intraperitoneal (IP), intravenous (IV) or subcutaneous injection (SubQ)), transdermal (either passively or using iontophoresis or electroporation), or transmucosal (nasal, vaginal, rectal, or sublingual) routes of administration. The formulation can be in the form of a suspension or emulsion. In general, pharmaceutical compositions can include pharmaceutically acceptable diluents, preservatives, solubilizers, emulsifiers, adjuvants and/or carriers. Such compositions can include diluents, sterile water, buffered saline of various buffer content (e.g., Tris-HCl, acetate, phosphate) at various pHs and ionic strengths; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate.

The formulations may be lyophilized and redissolved/resuspended immediately before use. The formulation may be sterilized by, for example, filtration through a bacterium retaining filter, by incorporating sterilizing agents into the compositions, by irradiating the compositions, or by heating the compositions.

In some embodiments, particularly those in which active agents are tethered thereto, the scaffolds may be administered to a subject in need thereof to treat a disease or disorder. In some embodiments, the compositions are administered systemically, for example, by intravenous or intraperitoneal administration, in an amount effective for delivery of the compositions to targeted cells. Other possible routes include, but are not limited to, trans-dermal and oral.

In certain embodiments, the compositions are administered locally, for example, by injection directly into a site to be treated. For example, in some embodiments such as for the treatment of cancer, the compositions are injected or otherwise administered directly to one or more tumors. Typically, local injection causes an increased localized concentration of the compositions which is greater than that which can be achieved by systemic administration. In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems: Fundamentals and Techniques (Chichester, England: Ellis Horwood Ltd., 1988 ISBN-10: 0895735806), which can affect a sustained release of the drug to the immediate area of the implant.

The compositions can be provided to the subject or cells either directly, such as by contacting it with the subject or cells, or indirectly, such as through the action of any biological process. For example, the scaffolds can be formulated in a physiologically acceptable carrier, and injected into a tissue or fluid surrounding the cell.

As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally, dosage levels of 0.001 to 10 mg/kg of body weight daily are administered to mammals. Generally, for intravenous injection or infusion, dosage may be lower. Generally, the total amount of the active agent administered to an individual using the disclosed vesicles can be less than the amount of unassociated active agent that must be administered for the same desired or intended effect and/or may exhibit reduced toxicity.

### III. Methods of Use

Glycans are abundantly expressed on surfaces of all cells. The proteins, glycan-binding proteins (GBPs), that bind cell surface glycans are also typically expressed on the surface of cells. The binding interaction of a cell surface glycan ligand with its corresponding cell surface GBP is therefore a multivalent interaction because many copies, i.e., hundreds to tens of thousands identical copies, of each distinct glycan structure and each distinct GBP will be present on cell surfaces. Such multivalent interactions result in a substantial increase in apparent binding affinity (avidity) due to “forced proximity” (Vauquelin, et al., Br J Pharmacol. 168(8):1771-85 (2013). doi: 10.1111/bph.12106); the high local “target” concentrations experienced by unbound glycan ligand or GBP once one (or more) glycan ligand(s) has bound to complementary GBP(s). Forced proximity significantly increases binding/rebinding of glycan:GBP complexes and substantially slows dissociation of the interaction, because all glycan:GBP complexes must dissociate simultaneously. This multivalent avidity enhancement increases apparent affinity many thousands-fold versus monovalent binding and accounts for the difficulty with inhibiting glycan:GBP-mediated cell-cell interactions despite the poor monovalent affinities that typify glycan:GBP binding.

The disclosed multivalent glycan scaffolds solve this problem. As illustrated with the experimental data below that is the case, i.e., monomer H3 binding to anti-H3 antibody is not detected by flow cytometry, and furthermore, that multimerization of the H3 glycan with the disclosed scaffolds yields a multimer H3:anti-H3 antibody complex that is easily detected by flow cytometry.

Thus, methods of using the scaffolds to identify binding interaction of specific glycan:GBP to e.g., identify glycan:GBP binding partners, understand the structural/chemical determinants of binding specificity/discrimination, isolate both GBPs that bind specified glycan structures and glycans that bind specified GBPs, and/or target cells for diagnostic detection and/or therapeutic elimination/inhibition or activation that express a GBP(s) having specificity for a particular glycan structure are provided.

For example, scaffold structures that include a detection tag, e.g., fluorophore, and/or an identifier tag, e.g. unique DNA sequence associated with each distinct glycan, may be used to identify those cells and tissues to which each distinct glycan directs localization of the structure in vivo. This can facilitate the design and delivery of cell/tissue-specific targeting.

These structures are therefore useful to (a) identify and isolate receptors for the ligands of interest, (b) identify cells and tissues that express those receptors, (c) target cells and tissues that express receptors for the ligand(s) to induce a signaling event or deliver a therapeutic entity, including a gene or material that modifies a gene, and (d) deliver antigens to specific cell types capable of processing the antigen for inducing optimal immunity, e.g., dendritic cells, or inducing antigen-specific tolerance, e.g., glycan ligands that target CD22 on B lymphocytes.

Methods and steps used in the identification and analysis and subsequent application or use of glycan binding events are provided in more detail below.

A. Contacting Glycan Scaffold with Target Material

Typical methods can include contacting one or more species (e.g., a library) of multivalent glycan scaffolds with a target material such as a population cells or proteins, e.g., a lysate as introduced above. In some embodiments, either the scaffold or the target material is immobilized at the time of contacting. In other embodiments, both the scaffold and the target material are free or mobile, e.g., in solution.

Either the scaffold glycans or the target material or both can be used as bait. Thus, in some embodiments, known or unknown cell type(s) or GBP(s) is used as bait to capture known or unknown glycan binding partner(s) from a library of glycan scaffolds. In other embodiments, glycan scaffolds with known or unknown glycan(s) are used to capture known or unknown cells or GBPs from a heterogenous population of cells or proteins (e.g., a lysate). FIG. 1C is a flow diagram of a library of multimeric ligand particles (MLPs) each presenting a distinct glycan and DNA or RNA sequence identifier (barcode) that identifies glycans that target specific cells and/or tissues.

Thus, embodiments are provided in which one of bait or the target is unknown and the other is unknown, both the bait and the target are known, and both the bait and the target are unknown.

In an embodiment, a library of multimeric glycan scaffolds is barcoded such that each scaffold displays a distinct multimeric glycan and a distinct barcode that uniquely identifies that glycan. This library is exposed to (e.g., a mixture of) cells (e.g., human blood cells) along with a mixture cell type-specific antibodies with each cell type-specific antibody labeled with a distinct fluorophore such that individual blood cell types can be separated and collected by FACS. PCR amplification and sequences of the barcode in each collected cell type “pool” will identify which cell types express GBPs for the identified glycans. This strategy can also be used for a mixture of GBPs provided when paired with reagents, e.g., anti-GBP antibodies, or distinct chemical or recombinant tags for each GBP. In vivo the anatomical location of the cell/tissue would identify the presence of GBPs associated with that cell/tissue responsible for the localization of the multimeric glycan scaffold. Additionally, the methods may assist in identifying those glycan structures that result in intracellular uptake of the multimeric glycan scaffold, and such identified glycans can be utilized for targeting and intracellular delivery.

In some embodiments, where both the bait and the target are unknown, the method resembles the shotgun glycan microarray approach (e.g., described by Rick Cummings, notably with the Human Milk Oligosaccharides, see, e.g., Yu, et al., Mol Cell Proteomics, 13(11):2944-60 (2014). doi: 10.1074/mcp.M114.039875). Without knowing what the oligosaccharides were, they were fractionated by HLPC, printed on a microarray slide and then interrogated by neonatal rotaviruses. The process of printing/immobilizing ligand(s) on a solid phase material produces a multivalent presentation of that ligand(s). Positive spots (signaling interaction) could be further investigated by going back to the fraction of interest and applying standard glycan analytical methods to identify the glycan(s) in the bound fractions. With that, the virus lectin can also be investigated further now knowing the appropriate glycan ligand(s). A similar approach can be utilized using the disclosed glycan scaffolds. Fractionate a mixture of glycans, (e.g., N3) tag, generate glycan scaffolds (e.g., dendrimers) with a unique identifier (e.g., nucleic acid barcode), sort through a sample of cells/proteins and PCR to find the glycan dendrimer binders, and then return to the corresponding fraction and analyze its contents.

The contacting can be carried out in vitro or in vivo. For in vitro applications, the methods typically include contacting the glycan scaffolds and cells or protein or other target material, and allowing for a sufficient amount of time for the plurality of scaffolds to bind one or more elements of the target material. For in vivo applications, the methods typically include administering the glycan scaffolds to subject, allowing for a sufficient amount of time for the plurality of scaffolds to bind one or more target GBPs on one or more cells in the subject and optionally obtaining one or more cells from the subject. Exemplary in vivo embodiments including imaging, diagnostic, and therapeutic applications are discussed below.

### B. Identification and Analysis of Binding Events

After a suitable period to allow binding between scaffold glycans and one or more elements of the target material, and one or more optional washing steps, binding events can be analyzed. For example, separation tags can be used to isolate scaffolds, label tags can used to detect or isolate scaffolds by, e.g., staining, flow cytometry, fluorescence activated cell sorting (FACS), magnetic-activated cell sorting (MACS), ELISA, immunohistochemistry, etc., optionally, but preferably bring their captured target elements along through the isolation or analysis.

### 1. Identification and Analysis of Glycan Scaffolds

Any of the methods can include sequencing a scaffold barcode to determine specific information about the (e.g., isolated or collected) scaffolds, such as the nature of underlying scaffold, glycan(s), and functional elements. This can, for example, facilitate identification of unknown glycans binding to known cells or proteins. Optionally, the barcode sequence is amplified. One of skill in the art knows how to design amplification primers that specifically amplify the barcode identifier sequence and/or the unique molecular identifier sequence. Optionally, the amplification primers are designed so that detection of an amplified barcode identifier sequence is sufficient to identify the barcode identifier sequence, thereby identifying the scaffold. In certain embodiments, the amplification is performed using polymerase chain reaction (PCR), for example, quantitative PCR.

Optionally, the one or more amplified barcode sequences and/or unique molecular identifier sequences are quantified. By determining the number of one or more barcode identifier sequences, one can determine whether the scaffold can bind to a specific tissue or organ and/or assess the efficacy or specificity or frequency of binding to one or more cell types, tissues or organs.

The barcode sequences and/or the unique molecular identifier sequences are optionally sequenced. Sequencing methods include, but are not limited to, shotgun sequencing, bridge PCR, Sanger sequencing (including microfluidic Sanger sequencing), pyrosequencing, massively parallel signature sequencing, nanopore DNA sequencing, single molecule real-time sequencing (SMRT) (Pacific Biosciences, Menlo Park, Calif.), ion semiconductor sequencing, ligation sequencing, sequencing by synthesis (Illumina, San Diego, Ca), Polony sequencing, 454 sequencing, solid phase sequencing, DNA nanoball sequencing, heliscope single molecule sequencing, mass spectroscopy sequencing, pyrosequencing, Supported Oligo Ligation Detection (SOLiD) sequencing, DNA microarray sequencing, RNAP sequencing, tunneling currents DNA sequencing, and any other DNA sequencing method identified in the future. One or more of the sequencing methods described herein can be used in high throughput sequencing methods. As used herein, the term high throughput sequencing refers to all methods related to sequencing nucleic acids where more than one nucleic acid sequence is sequenced at a given time. The amplification products can be pooled prior to sequencing.

In preferred embodiments, the barcode encodes the identification of, or can be otherwise used to determine, the composition of the glycan scaffold associated therewith. See also (Sago, et al., Proc Natl Acad Sci USA., 115(42):E9944-E52. Epub 2018/10/03. doi: 10.1073/pnas.1811276115. PubMed PMID: 30275336; PMCID: PMC6196543 (2018)) and U.S. Published Application No. 2020/0110083.

2. Identification and Analysis of Target Material Bound target materials such as cells and/or GBPs can be identified and/or analyzed. For example, target proteins can be subject to protein analysis and/or identification such as Western blotting (e.g., immunoblotting), Edman degradation, mass spectrometry including peptide mass fingerprinting or de novo peptide sequencing, light scattering such as Batch Dynamic Light Scattering (DLS), Static Light Scattering (SLS), Charge and Zeta Potential, Multi-Detection GPC/SEC, Circular Dichroism (CD), or Isothermal titration calorimetry (ITC). Target cells can be identified or characterized by one or more protein or gene expression analysis, phenotypic and/or histological analysis by, for example, microscopy including light, fluorescent and/or, electron microscopy. In some embodiments, the target material is released from the scaffold prior to or during the identification and/or analysis. In other embodiments, the target material is not released from the scaffold. In such embodiments, the scaffold and/or bait, etc., can optionally be subtracted from the analysis. For example, when mass spectrometry, the signal generated by the scaffold and/or bait can be subtracted from the spectrograph.

Analysis of the target material may also include assaying for induction and/or inhibition of cellular pathways (e.g., signaling pathways) that are modulated by glycan of the scaffold that binds thereto.

### C. Applications and Therapeutic Methods

Once binding relationships between glycans and target material such as cells or proteins are established, scaffolds featuring the relevant glycans can be rationally designed for targeted delivery of one or more active agents to the cell(s), tissue(s), organ(s), and/or protein(s).

### 1. Targeted Delivery of Active Agents Using Glycan Scaffolds

In preferred embodiments, the analysis reveals that one or more glycan scaffolds preferentially bind to specific target proteins or cells. Preferentially binds means that more of a particular glycan scaffold species binds to one proteins or cell type(s) as compared to other proteins or cell type(s). For example, such preferential binding may be across organs or tissues (e.g., liver instead of pancreas), across pathologic/normal cells (e.g., cancer versus non-cancerous), or across cells within the same tissue or organ (e.g., lymphocytes as opposed to monocytes). By identifying scaffolds with preferentially binding, one of skill in the art can identify targeted scaffolds that, when administered to the subject, preferentially deliver their functional element, for example, tags, labels, or active agents, to a particular cell type or a particular tissue or organ of the subject. Depending on the tissue or organ, once scaffold binding events are identified to a desired cell type(s), one of skill in the art can select or prepare targeted glycan scaffolds that are functionalized with one or more active agents for delivery the cell(s), tissue(s), or organ(s) in the subject. The active agent is selected based on the desired therapy, which as introduced above, can include delivery of small molecule, protein, and nucleic acid therapeutics, vaccine components such as antigens and/or adjuvants, etc. Thus, any of the methods can further include in vitro or in vivo delivery of one or more active agents.

In other embodiments, that analysis identifies that the glycans themselves induce or inhibit an action or activity of the cell (e.g., cellular signaling). In such embodiments, although a similar principle of preferential binding is used to identify a desired binding relationship and therapeutic outcome, the glycan scaffold may optionally lack functionalization with a further functional element (e.g., active agent such as a drug, or a tag, etc.), because the glycan itself is the active agent that modulate the therapeutic result (e.g., activation or inhibition).

Thus, in vivo methods may include, for example, administering a subject in need thereof a pharmaceutical composition containing an effective amount of a glycan scaffold optionally with an active agent functionalized thereto.

In other embodiments, glycan scaffolds in suspension or immobilized on a substrate are used in place of, or as an adjunct to, plasmapheresis. For example, once a GBP (i.e., an IgG, etc.) from blood or another fluid is identified, the corresponding glycan scaffold can be used to remove the GBP from the blood or other fluid by contacting the blood or other fluid with the glycan scaffold, and then separating the glycan scaffold from the blood or other fluid. The GBP binds to the glycan scaffold and remains bound thereto when the blood or other fluid is removed. Such methods can be carried out ex vivo as in traditional plasmapheresis, or in vivo, typically wherein the GBP-bound glycan scaffold is subsequently collected so the GBP can be removed.

Similarly, imaging methods may include administering a subject in need thereof a pharmaceutical composition containing a glycan scaffold with an imaging agent functionalized thereto (e.g., luciferase substrate tag or fluorophore on the dendrimer). Methods of detecting a target material in a subject may include imaging the subject, and determining or locating the target material in the subject when and/where the glycan scaffold is detected. If the target material is indicative of a disease or disorder, the subject can be subsequently diagnosed with the disease or disorder.

### 2. Exemplary Use Strategies

Exemplary, non-limiting use strategies are also provided.

For example, glycan (self or from pathogens) can be used to stimulate innate and adaptative immune system via, e.g., Siglecs (Murugesan, et al., Curr Opin Chem Biol., 62:34-42, doi: 10.1016/j.cbpa.2021.01.001 (2021)) and C-type lectins (Richardson, et al., Front Immunol., 5:288, doi: 10.3389/fimmu.2014.00288 (2014)) such DC-SIGN and MGL. Pathogen interactions/recognitions with host cells are often glycan dependent. Glycan scaffolds can be used to explore, define, and neutralize these interactions. Several works have studied these possibilities using di-/tri-saccharides (Cordoba, et al., Nanomedicine, 9(7):972-84, doi: 10.1016/j.nano.2013.03.004 (2013), Clayton, et al., Bioconjug Chem., 22(10):2186-97, doi: 10.1021/bc200331v (2011), Garcia-Oliva, et al., Chemistry, 26(7):1588-96, doi: 10.1002/chem.201903788 (2020), Bernardi et al., Chem Soc Rev., 42(11):4709-27, doi: 10.1039/c2cs35408j (2013), Kensinger, et al., Antimicrob Agents Chemother, 48(5):1614-23, doi: 10.1128/AAC.48.5.1614-1623.2004 (2004), Imberty, et al., Chemistry, 14(25):7490-9, doi: 10.1002/chem.200800700 (2008), Hoyos, et al., Chemistry, 27(28):7593-624, doi: 10.1002/chem.202005065 (2021), Gingras, et al., Chem Soc Rev., 42(11):4823-41, doi: 10.1039/c3cs60090d (2013), Wells, et al., Adv Ther (Weinh), 2021:2000210, doi: 10.1002/adtp.202000210 (2021), Varga, et al., Biomaterials, 35(13):4175-84, doi: 10.1016/j.biomaterials.2014.01.014 (2014), Mintzer, et al., Mol Pharmm., 9(3):342-54, doi: 10.1021/mp2005033 (2012)). With the possibility to using full length, complex N-glycans on the disclosed glycan dendrimers new studies can be performed, with host cell N-glycans/O-glycans for instance.

Cell-surface glycan binding protein receptors can be targets for intracellular drug delivery/targeting. See, e.g., Cattiaux, et al., Bioorg Med Chem., 26(5):1006-15, doi: 10.1016/j.bmc.2017.12.036 (2018), Krist, et al., Biochem Biophys Res Commun., 287(1):11-20, doi: 10.1006/bbrc.2001.5537 (2001), Bernardes, et al., Org Biomol Chem., 8(21):4987-96. Epub 2010/09/08. doi: 10.1039/c0ob00372g (2010), Bezouska, et al., FEBS Lett., 426(2):243-7, doi: 10.1016/s0014-5793(98)00340-8 (1998), Thoma, et al., Chemistry, 12(1):99-117. Epub 2005/10/19. doi: 10.1002/chem.200500901 (2005), Lepenies, et al., Adv Drug Deliv Rev., 65(9):1271-81, doi: 10.1016/j.addr.2013.05.007 (2013), Doores, et al., Chemistry., 12(3):656-65, doi: 10.1002/chem.200500557 (2006), Zaal, et al., Front Immunol., 11:305, doi: 10.3389/fimmu.2020.00305 (2020). In particular embodiments, the compositions and methods are used to target cancer cells expressing a particular ligand for the glycan of the glycan scaffold. In other embodiments, the compositions and methods are used to reduce inflammation.

The invention can be further understood by the following numbered paragraphs:

1. A method of identifying glycan-based binding interactions comprising contacting a target material with a known multivalent glycan scaffold comprising two or more glycans, and identifying element(s) from the target material that binds thereto.

2. The method of paragraph 1, wherein the target material is a population of cells comprising one more different cell types, or a cell lysate or other proteinaceous mixture comprising one or more glycan binding proteins (GBP).

3. The method of paragraph 2, wherein the target material is a population of cells comprising two more different cell types, or a cell lysate or other proteinaceous mixture comprising two or more glycan binding proteins (GBP).

4. The method of paragraph 3, wherein the target material is a population of cells and the target material element(s) is one or more of the cell types therein.

5. The method of paragraph 4, wherein the one or more cell types preferentially binds to the multivalent glycan scaffold compared to other cell type(s) in the population.

6. The method of any one of paragraphs 2-5, further comprising isolating the multivalent scaffold with the cells bound thereto.

7. The method of any one of paragraphs 2-6 further comprising characterizing the bound cells.

8. A composition comprising the glycan scaffold-bound cells of any one of paragraphs 2-7.

9. The method of paragraph 2, wherein the target material is a cell lysate or other proteinaceous mixture and the target material element is one or more of the GBPs therein.

10. The method of paragraph 9, wherein the one or more GBPs preferentially binds to the multivalent glycan scaffold compared to other GBPs in the cell lysate or other proteinaceous mixture.

11. The method of any one of paragraphs 9 or 10 further comprising characterizing the bound GBP.

12. The method of any one of paragraphs 9-10 further comprising isolating the bound GBP.

13. A composition comprising the glycan scaffold-bound GBP of any one of paragraphs 9-12.

14. A method of identifying glycan-based binding interactions comprising contacting one or more known target material elements with one or more species of multivalent glycan scaffolds each comprising two or more glycans, and identifying glycan scaffold(s) that bind thereto.

15. The method of paragraph 14 comprising contacting the one or more known target material elements with a library of different species of multivalent glycan scaffolds each comprising two or more glycans and identifying glycan scaffold(s) that bind thereto, optionally wherein the library comprises between about 2 to about 10,000 different species of glycan scaffolds, or any subrange or specific integer thereof, such as 2, 5, 10, 25, 50, 75, 100,250, 500, 1,000.

16. The method of paragraph 14 or 15, wherein the known target material element is one or more cell types.

17. The method of paragraph 16, wherein one or more of the multivalent glycan scaffolds preferentially bind to the one or more cell types compared to other multivalent glycan scaffolds in the library.

18. The method of any one of paragraphs 16 or 17 further comprising characterizing the bound multivalent glycan scaffold(s).

19. The method of any one of paragraphs 16-18 further comprising isolating the multivalent glycan scaffold(s).

20. A composition comprising the glycan scaffold-bound cells of any one of paragraphs 14-19.

21. The method of paragraphs 14 or 15, wherein the known target material element is one or more GBP.

22. The method of paragraph 21, wherein one or more of the multivalent glycan scaffolds preferentially bind to the one or more GBP compared to other multivalent glycan scaffolds in the library.

23. The method of any one of paragraphs 21 or 22 further comprising characterizing the bound multivalent glycan scaffold(s).

24. The method of any one of paragraphs 21-23 further comprising isolating the multivalent glycan scaffold(s).

25. A composition comprising the glycan scaffold-bound GBP of any one of paragraphs 21-24.

26. A method of identifying glycan-based binding interactions comprising contacting a target material with one or more unknown multivalent glycan scaffolds comprising two or more glycans, and identifying element(s) from the target material that binds thereto, optionally wherein the one or more unknown multivalent glycan scaffolds form a library comprising between about 2 about 10,000 different species of glycan scaffolds, or any subrange or specific integer thereof, such as 2, 5, 10, 25, 50, 75, 100, 250, 500, 1,000.

27. The method of paragraph 26, wherein the target material is a population of cells comprising one more different cell types, or a cell lysate or other proteinaceous mixture comprising one or more glycan binding proteins (GBP).

28. The method of paragraph 27, wherein the target material is a population of cells comprising two more different cell types, or a cell lysate or other proteinaceous mixture comprising two or more glycan binding proteins (GBP).

29. The method of paragraph 28, wherein the target material is a population of cells and the target material element(s) is one or more of the cell types therein.

30. The method of paragraph 29, wherein the one or more cell types preferentially binds to the multivalent glycan scaffold(s) compared to other cell type(s) in the population.

31. The method of any one of paragraphs 26-30, further comprising isolating the multivalent scaffold(s) with the cells bound thereto.

32. The method of any one of paragraphs 26-31 further comprising characterizing the bound cells and/or the glycan(s) bound thereto.

33. A composition comprising the glycan scaffold-bound cells of any one of paragraphs 27-32.

34. The method of paragraph 27, wherein the target material is a cell lysate or other proteinaceous mixture and the target material element is one or more of the GBPs therein.

35. The method of paragraph 34, wherein the one or more GBPs preferentially binds to the multivalent glycan scaffold compared to other GBPs in the cell lysate or other proteinaceous mixture.

36. The method of any one of paragraphs 34 or 35 further comprising characterizing the bound GBP and/or the glycan(s) bound thereto.

37. The method of any one of paragraphs 34-36 further comprising isolating the bound GBP.

38. A composition comprising the glycan scaffold-bound GBP of any one of paragraphs 34-37.

39. The method or composition of any one of paragraphs 1-38, wherein the multivalent glycan scaffold comprises an underlying branched scaffold with two or more glycans and optionally one or more functional elements linked to the terminal ends thereof.

40. The method or composition of paragraph 39, wherein the underlying branched scaffold is a dendrimer.

41. The method or composition of paragraphs 39 or 40, wherein the two or more glycans are all of the same species of glycan.

42. The method or composition of paragraphs 39 or 40, wherein the two or more glycans are comprised of two or more different species of glycans.

43. The method or composition of any one of paragraphs 39-42, wherein one or more of the functional element(s) is an agent that facilitates formation, imaging, localization, identification, isolation, and/or characterization of the glycan scaffold, and/or the target material element bound thereto.

44. The method or composition of any one of paragraphs 39-43, wherein at least one of the functional elements is a nucleic acid comprising a barcode that specifically identifies the glycan scaffold.

45. The method or composition of any one of paragraphs 39-44, wherein at least one of the functional elements is a fluorophore.

46. The method or composition of any one of paragraphs 39-45, wherein the glycan is linked to 2-amino-N-(2-aminoethyl)-benzamide (AEAB) which is directly or indirectly linked to the terminal ends of the underling branched scaffold.

47. The method or composition of any one of paragraphs 39-45, wherein at least one of the functional elements is an affinity tag.

48. The method or composition of any one of paragraphs 39-47, wherein the glycan is linked to an amino acid which is directly or indirectly linked to the terminal ends of the underlying branched scaffold.

49. The method of paragraph 48, wherein the amino acid is no part of a polypeptide chain.

50. The method of paragraph 48, wherein the amino acid is part of a polypeptide chain.

51. The method of paragraph 50, wherein the polypeptide chain comprises two or more amino acid acids with glycans linked thereto.

52. The method of any one of paragraphs 48-51, wherein the amino acid is serine or threonine.

53. The method or composition of any one of paragraphs 1-52, wherein the glycan scaffold or target material element is immobilized, optionally to a surface or substrate such as bead, a slide, a plate, or a dish.

54. The method or composition of any one of paragraphs 1-52, wherein neither the glycan scaffold or target material are immobilized.

55. The method or composition of paragraph 54, wherein the glycan scaffold and target material are in solution.

56. A method of treating a subject in need thereof, comprising identifying a glycan-based binding interaction according to any of the preceding methods and administering the subject a composition comprising an effective amount of a therapeutic multivalent glycan scaffold comprising the glycan of the identified interaction.

57. The method of paragraph 56, wherein therapeutic glycan induces or inhibits a cellular activity upon binding to cell or GBP of the interaction.

58. The method of paragraph 56, wherein therapeutic glycan comprise an active agent functionalized thereto.

59. The method of paragraph 58, wherein the active agent is a nucleic acid, small molecule, or polypeptide.

60. The method of paragraph 59, wherein the cell is a cancer cell and the active agent is an anti-cancer drug.

61. The method of paragraph 59, wherein the cell is an immune cell and the active agent is or encodes an antigen.

62. A composition comprising the therapeutic glycan of any one of paragraphs 56-62.

63. A multivalent glycan scaffold comprising an underlying branched scaffold with two or more glycans and one or more functional elements linked to the terminal ends thereof.

64. The multivalent glycan scaffold of paragraph 63, wherein at least one of the functional element(s) is an agent that facilitates formation, imaging, localization, identification, isolation, and/or characterization of the glycan scaffold, and/or a target material element bound thereto.

65. The multivalent glycan scaffold of any one of paragraphs 63 or 64, wherein at least one of the functional elements is a fluorophore.

66. The multivalent glycan scaffold of any one of paragraphs 63-65, wherein the glycan is linked to 2-amino-N-(2-aminoethyl)-benzamide (AEAB) which is directly or indirectly linked to the terminal ends of the underling branched scaffold.

67. The multivalent glycan scaffold of any one of paragraphs 63-66, wherein at least one of the functional elements is a nucleic acid comprising a barcode that specifically identifies the glycan scaffold.

68. The multivalent glycan scaffold of any one of paragraphs 63-67, wherein at least one of the functional elements is an affinity tag.

69. The method of composition of any one of paragraphs 63-68, wherein the glycan is linked to an amino acid which is directly or indirectly linked to the terminal ends of the underlying branched scaffold.

70. The method of paragraph 69, wherein the amino acid is no part of a polypeptide chain.

71. The method of paragraph 69, wherein the amino acid is part of a polypeptide chain.

72. The method of paragraph 71, wherein the polypeptide chain comprises two or more amino acid acids with glycans linked thereto.

73. The method of any one of paragraphs 69-72, wherein the amino acid is serine or threonine.

74. The multivalent glycan scaffold of any one of paragraphs 63-73, wherein the underlying branched scaffold is a dendrimer.

75. The multivalent glycan scaffold of any one of paragraph 63-74, wherein the underlying scaffold is formed of 2-16 arms, inclusive, optionally 8-16 arms.

76. The multivalent glycan scaffold of any one of paragraph 63-75, wherein the underlying scaffold and/or arms thereof is formed of polyethylene glycol (PEG) polymer.

77. The multivalent glycan scaffold of any one of paragraphs 63-76 wherein the underlying scaffold is formed of 8-Arm PEG-Biotin/Alkyne, Biotin(1)/Alkyne(7), MW 20 k; Dendro Alkyne-PEG-Dendro Alkyne, PEG 10 k, G4 32 Alkyne; Dendro Alkyne-PEG-Dendro Alkyne, PEG 10 k, G5 64 Alkyne; or Dendro Alkyne-PEG-Dendro Alkyne, PEG 10 k, G6 128 Alkyne.

78. The multivalent glycan scaffold of any one of paragraphs 63-77, wherein the multimeric glycan scaffold is between 10 kDa and 50kD, or between 20 kDa and 60 kDa, or between 20 kDa and 30 kDa, each inclusive, or any integer range thereof, or any specific integer therebetween.

79. The multivalent glycan scaffold of any one of paragraphs 63-78, wherein the two or more glycans are all of the same species of glycan.

80. The multivalent glycan scaffold of any one of paragraphs 63-79, wherein the two or more glycans are comprised of two or more different species of glycans.

81. The multivalent glycan scaffold of any one of paragraphs 63-80 comprising the formula:

(Ligand)n:Scaffold:Detection/Separation Tag:Identifier Tag
wherein “Ligand” is present and is one or more species of glycan having n >1 copies (i.e., multivalent);
“Scaffold” is present and is the underlying branched scaffold, e.g., dendrimer or hyperbranched polymer;
one or both of “Detection/Separation Tag” and “Identifier Tag” are present and refer to the functional element e.g., (a) biotin or (HIS)6-8 peptide tag to facilitate separation/isolation, (b) a fluorophore to facilitate detection, (c) a unique DNA or RNA sequence (“barcode”) that can be PCR amplified and sequenced to facilitate identification; and/or (d) a drug or other material(s) as discussed elsewhere herein, e.g., nucleic acids such as DNA, RNA, iRNA, crispr guide RNA, etc., or vesicle containing a drug or such other materials that are intended to have a therapeutic or cell modifying effect or can include an antigen that is intended to induce (a) a protective immune response directed to a pathogen or an aberrant cell, e.g., tumor, that expresses the antigen or (b) immune tolerance to the antigen as a therapy for an autoimmune disorder directed against the antigen.

82. The multivalent glycan scaffold of any one of paragraphs 63-81, wherein the glycan scaffold is not immobilized.

83. A multivalent glycan scaffold as described herein, including but not limited to, the text and/or figures.

84. A composition comprising a plurality of multivalent glycan scaffold of any of paragraphs 63-83 in solution.

85. The composition of paragraph 84, wherein the plurality of multivalent glycan scaffolds are not immobilized.

### EXAMPLES

### Example 1: Generation of Multivalent, Multifunction Glycan Dendrimers

### Material & Methods

### Reagents

2-chloro-1,3-dimethylimidazolinium chloride (DMC), Sigma Aldrich (#529249-5G)
Sodium azide (NaN3), Fisher Scientific (#BP922I-500)
2,6-lutidine, Sigma Aldrich (#336106-100ML)
Acetonitrile (ACN), Fisher Scientific (#AA42311K2)
Trifluoroacetic acid (TFA), Fisher Scientific (#AAL06374AC)
Carbograph column 150 mg, BGB analytik (#SPU-5122423)
PNGaseF, New England Biolabs (#P0704)
Blood group H-type 2-trisaccharide, Chemilly Glycoscience (#OS03006)
Lewis X tetrassacharide, Carbosynth (#OL09569)
3′-sialylLacNAc, Carbosynth (#OS00732)
6′-sialylLacNAc, Dextrauk (#SLN306)
Globo-N-pentaose (Gb5), Chemily Glycoscience (#OS03012)
Globo-N-tetraose (Gb4); Elicityl (#GLY121-90%)
8-mer PEG dendrimers (7 arms alkyne-functionalized; 1 arm biotin-functionalized), Creative PEGWorks (#CPW-8841)
Multivalent H3-PAA-biotin, Glycotech (#01-034)
Monovalent H3-sp-biotin, Glycotech (#02-034)
Multivalent LewisA-PAA-biotin, Glycotech (#01-035)
Jurkat cells, Ipracell (#CC-05-10-50)
Bovine fetuin, MyBioSource (#MBS634403)
Chicken ovalbumin, Fisher Scientific (#50-136-8468)
Ribonuclease B (RNAseB), Worthington (#LS005710)
Human IgG, Athens Research & Technology (#16-16-090707)
Porcine Stomach Mucin (PSM), Sigma Aldrich (#M2378-100G)
CuSO₄, Sigma Aldrich (#451657-10G)
Tris-hydroxypropyltriazolylmethylamine (THPTA), Fisher Scientific (#NC1486137)
Sodium ascorbate, Sigma Aldrich (#11140-50G)
Sodium phosphate buffer (PBS), 100 mM monosodium phosphate, RPI (#S23185-500.0), 100 mM disodium phosphate, RPI (#S23175-500.0), pH 7.4
PD-10 Sephadex G-25 desalting column, Cytiva (#17085101)
TRIS, RPI (#T60040-1000.0)
NaCl, RPI (#S23025-3000.0)
CaCl₂, RPI (#C36200-500.0)
MgCl₂, RPI (#M24000-100.0)
Sodium carbonate anhydrous, RPI (#25025-1000.0)
Sodium bicarbonate, RPI (#S22060-1000.0)
Bovine serum albumin (BSA), RPI (#A30075-100.0)
Streptavidin-PE, Invitrogen (#SNN1007)
Propidium Iodide (PI), Mitenyi Biotec (#130-093-233)
Neutralite Avidin-HRP, SouthernBiotech (#7200-05)
TMB ELISA Substrate, AbCan (#ab171524)

### Azide Derivatization of Glycans

Adapted from Tanaka, et al., Chem Commun (Camb), (23):3378-9, doi: 10.1039/b905761g (2009) and Streichert, et al., Chembiochem., 20(15):1914-8, doi: 10.1002/cbic.201900023 (2019), which are specifically incorporated by reference herein in their entireties. Commercial glycans, including Blood group H type 2 trisaccharide, Lewis X tetrassacharide, 3′-and 6′-sialylLacNAc, Globo-N-pentaose (Gb5), and Globo-N-tetraose (Gb4) were derivatized at a concentration of 50 mM in a final volume of 45 to 60 μL. The glycans were initially solubilized in a minimal volume (10 to 15 μL) of MilliQ water to which a solution of NaN3 and 2,6-Lutidine was added to a final concentration of 3.5 M and 1.5 M, respectively. DMC was added at a final concentration of 750 mM and the final solution was incubated for 72 h at 4° C. with occasional mixings to homogenize the sample. See, Table 1.

TABLE 1

| H type 2 trisaccharide | | | | | | | |
|---|---|---|---|---|---|---|---|
| (H3) (~530 Da) | Eq. | Quantity | | Moles | | Concentratior | |
| H3 | 1 eq | 1.32 | mg | 2.5 | μmol | 50 | mM |
| DMC | 15 eq | 6.34 | mg | 37.5 | μmol | 750 | mM |

| | | | | | | |
|---|---|---|---|---|---|---|
| 2,6-lutidine | 30 eq | 8.97 | μL | 74.8 | μmol | 1.5M |
| NaN3 | 70 eq | 11.33 | mg | 174.3 | μmol | 3.5M |

Final volume 50 μL.

The N3-derivatized glycans were purified over a carbograph column (150 mg capacity) conditioned with 1 Column Volume (CV) of ACN+0.1% TFA, 1 CV of MilliQ water+0.1% TFA, 1 CV of 10% ACN+0.1% TFA, and 1 CV of MilliQ water+0.1% TFA. 1 mL of MilliQ water+0.1% TFA was added to the glycans azidation reaction solution and then loaded onto the carbograph column. The reaction tube was rinsed with 1 mL of 10% ACN+0.1% TFA and loaded onto the carbograph column. The carbograph column was then washed with 3 CV of 10% ACN+0.1% TFA. The glycans were eluted with 2 CV of 25% ACN+0.1% TFA followed by 1 CV of 50% ACN+0.1% TFA. Fractions were pooled and lyophilized.

For larger, N-glycan-type glycans (MW ˜1,500 to 3,000 Da), the conditions were adjusted. Final concentrations/volumes/moles as shown in Table 2.

TABLE 2

| N-glycans | | | | | | |
|---|---|---|---|---|---|---|
| (~2,000 Da) | Eq. | Quantity | | Moles | Concentratior | |
| N-glycans | 1 eq | 4 | mg | 2 μmol | 35 | mM |
| DMC | 20 eq | 6.76 | mg | 40 μmol | 700 | mM |

| | | | | | |
|---|---|---|---|---|---|
| 2,6-lutidine | 40 eq | 9.5 | μL | 48 μmol | 1.4M |
| NaN3 | 80 eq | 10.4 | mg | 96 μmol | 2.8M |

Final volume 57.2 μL.

### Glycan Coupling to the 8-Arm PEG Biotin/Alkyne Linker

Glycan-N3 and 8-arm PEG Biotin/Alkyne linker were resuspended in PBS at 20 mg/mL and 15 mg/mL, respectively. Fresh solutions of 20 mM CuSO₄, 20 mM THPTA, and 50 mM sodium ascorbate were prepared in PBS. The coupling reaction was carried out with a 21-molar excess of glycan-N3 compared to 8-arm PEG Biotin/Alkyne and in the following conditions—glycan-N3 0.45 mM, 8-arm PEG-Biotin/Alkyne 9.45 mM, CuSO₄ 0.2 mM, THPTA 1 mM, and sodium ascorbate 5 mM in a final volume of 200 μL of sodium phosphate buffer. The reaction was incubated at RT, overnight with gentle rotation.

The conjugated glycan-dendrimers were purified on a PD-10 G-25 column equilibrated with 10 mL of sodium phosphate buffer. The glycan-dendrimer solution was loaded onto the column, and the column washed with 10 mL of sodium phosphate buffer. 1 mL fractions were collected and fractions containing the glycan-dendrimers were pooled and lyophilized. Dry 8-mer-H3 was reconstituted in sodium phosphate buffer at 1 mg/ml, aliquoted, and frozen at −20° C.

### Flow Cytometry

1-2·10⁷ O13 YSD cells were washed once in TSM-BSA buffer (20 mM Tris pH 7.4, 150 mM NaCl, 2 mM CaCl₂, 2 mM MgCl₂, 1% BSA) and were then incubated with 100 mM of either H3-PAA-biotin or H3-sp-biotin, and 200 mM of H3 glycan dendrimer or unmodified dendrimer in 250 μL of TSM-BSA at 4° C. for 1 h with gentle shacking. The O13 YSD cells were then washed 3 times with TSM-BSA prior to incubation with streptavidin-PE (1:400 dilution) in 250 μL of TSM-BSA at 4° C., for 30 min, in the dark with gentle shaking. The O13 YSD cells were then washed 3 times with ice-cold TSM-BSA, then resuspended in 250 μL TMS-BSA to which 1 μL of PI was added to stain live/dead cells. 125 μL of each sample were used for flow cytometry measurements.

### ELISA

Wells of a 96-well plate were coated with 200 μL of 0.05M carbonate-bicarbonate buffer pH 9.6 containing 2 μg/ml of O13-hIgG1 and incubated at 37° C. for 30 minutes. The wells were blocked with TSM+3% BSA for 1 h at RT, then washed 3 times with TSM+0.05% Tween 20 (TMS-T). The wells were incubated with 100 nM of either H3-PAA-biotin, H3 glycan dendrimer, LewisA-PAA-biotin, or unmodified dendrimer in 200 μL of TSM buffer for 1 h at RT. After 3 washes with TSM-T, the wells were incubated for 1 h at RT with 200 μL of TSM containing a 1:2000 dilution of Avidin-HRP. Signal for each well was revealed by addition of 150 μL of TMB and reading using a plate reader with a 630 nm filter.

### Results

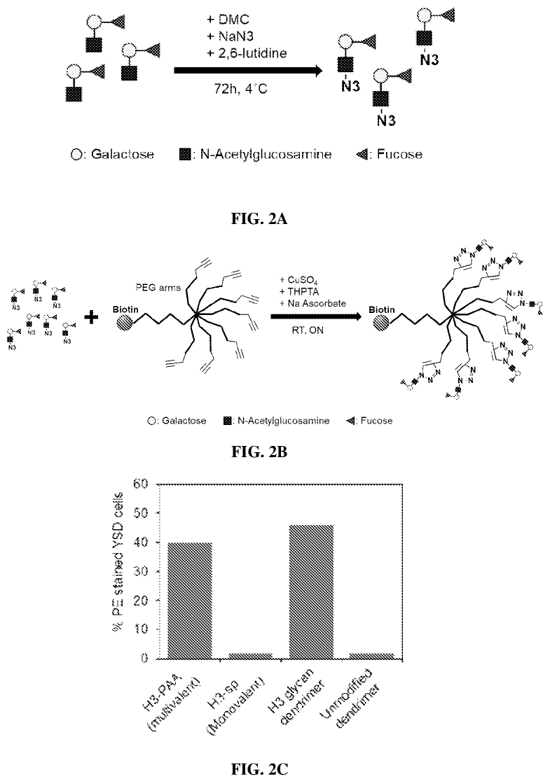

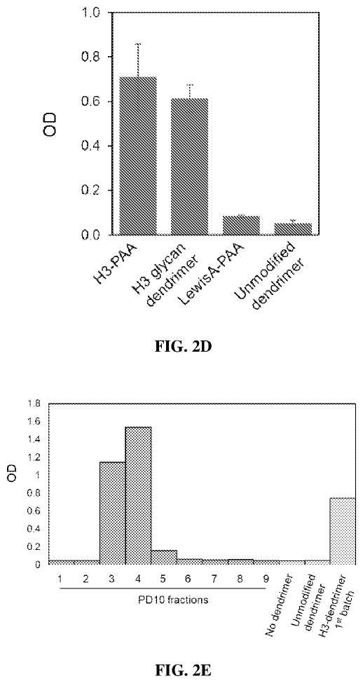

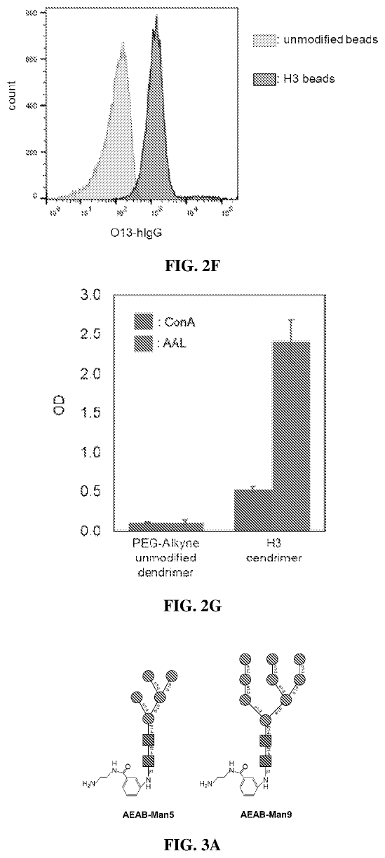

Blood H type 2 trisaccharides (H3) were derivatized with an azide (N3) functional group on C1 of the reducing monosaccharide, the N-acetylglucosamine in the case of H3 (FIG. 2A). The N³-tagged H3 glycans were “clicked” onto the alkyne arms of the 8-mer PEG Biotin/Alkyne dendriters (FIG. 2B).

The H3 glycan dendrimers were tested by flow cytometry with a yeast clone expressing the variable lymphocyte receptor (VLR) O13, a VLR highly specific for 113 (Collins, et al., Structure, 25(11):1667-78 e4, doi: 10.1016/j.str.2017.09.003 (2017)). Commercial multivalent H3-PAA-biotin was used as a positive control and has previously shown robust binding by O13 YSD cells. Commercial monovalent H3-sp-biotin and unmodified 8-mer PEG Biotin/Alkyne dendrimer were used as negative controls.

Results showed that H3-PAA-biotin and H3 glycan dendrimer were equally, if not higher for H3 glycan dendrimer, bound by O13 YSD cells. Both unmodified 8-mer PEG Biotin/Alkyne dendrimer and monovalent H3-sp-biotin showed no binding by O13 yeast (FIG. 2C).

The generation of H-trisaccharide (H3) dendrimers as initially described was fully repeated—azidation of H3, coupling to alkyne dendrimers and functional assays. All data were similar and reproducible. FIG. 2E shows the reactivity of PD10 fractions with the O13-hIgG anti-H3 antibody after conjugation of the PEG-Alkyne dendrimers with H3-N3. FIG. 2F shows the reactivity of O13-hIgG anti-H3 antibody to DBCO magnetic beads conjugated with or without H3-N3 (conjugated with H3-N3 only, not H3-dendrimers. This was done to demonstrate the N3 modification of H3). FIG. 2G indicates that H3-dendrimers coupled to magnetic beads are reactive to Aleuria Aurantia Lectin (AAL, specific for α-linked fucose) and unreactive to Concanavalin A (ConA, specific for mannose).

The results indicate that monovalent glycan (type 2 H trisaccharide antigen or H3) to an H3-specific GBP (anti-H3 antibody) cannot be detected by flow cytometry, whereas binding of the anti-H3 antibody GBP to multimer H3 made by the disclosed scaffolds is easily detected by flow cytometry. The dissociation constant (K_{d}) for the monomer H3:anti-H3 antibody interaction has been measured to be 2×10⁻⁶ M/L, which is considered a superior K_{d} for glycan:GBP interactions, i.e., many glycan:GBP K_{d} are >10⁻⁵ M/L and maybe as poor as 10⁻³ M/L. Since K_{d}=k_{off}/kₒₙ and kₒₙ for small molecules like glycans is >>10⁶ M⁻¹ s⁻¹, (see Fundamental Immunology 5th Edition, 2003 (William Paul) Chapter 4 Antigen-Antibody Interactions and Monoclonal Antibodies; Thermodynamics and Kinetics, Jay A. Berzofsky, Ira J. Berkower, Suzanne L. Epstein) then k_{off} will be 2 s⁻¹ for a K_{d}=2×10⁻⁶ M/L, and the half-life of the monomer H3:anti-H3 antibody complex will be t_{1/2}=ln 2/k_{off}=0.35 s. Such a transient complex is unlikely to survive the washes/incubations needed to remove unbound ligand (H3 glycan) and nonspecifically bound materials prior to detection of specifically bound ligand. As illustrated with the experimental data, that is the case, i.e., monomer H3 binding to anti-H3 antibody is not detected by flow cytometry, and furthermore, that multimerization of the H3 glycan with the disclosed scaffolds yields a multimer H3:anti-H3 antibody complex that is easily detected by flow cytometry.

In addition to H3, several other glycans including Lewis X tetrassacharide, 3′- and 6′-sialylLacNAc, Globo-N-pentaose (Gb5), and Globo-N-tetraose (Gb4) have been azide-derivatized, and used to generate the corresponding glycan dendrimers.

N-glycans from bovine fetuin, RNAse B, chicken ovalbumin and the human T lymphocyte cells Jurkat have been enzymatically released and will be used to confirm this approach can be used with naturally released glycans.

In sum, these experiments exemplify the derivatization and incorporation of an azide tag on the H type 2 trisaccharide (H3) and subsequent generation of an H3 glycan dendrimer using click chemistry. Flow cytometry binding experiments showed that H3 glycan dendrimer, but not the unmodified 8-mer PEG linker, was effectively bound by O13 YSD cells to a similar level as commercial H3-PAA. These results support use of this strategy as a platform for derivatization and display in a multivalent fashion of any glycan (e.g., with a reducing end), including commercially available, synthetic, or naturally released glycans. Monovalent glycans only have very weak interactions with their glycan-binding partners and, like in nature, multivalent glycan presentation is required to support robust binding. The provided multivalent glycan dendrimers can be used to, for example, screen for new glycan-binding molecules in any biological systems, to inhibit or activate glycan-dependent cell-surface receptors, and block glycan-dependent pathogen infections.

These experiments illustrate glycan dendrimers supporting multimeric display of glycans with a biotin-functionalized linker. Glycans with a reducing end from any sources can be functionalized and couple to an 8-mer-biotin linker. These strategies provide an easy and adaptable alternative to the multivalent glycan polymers currently commercially available.

The biotin handle provides experimental flexibility. For example, fluorescent tags can be added for flow cytometry/FACS and imaging experimentations. Glycan dendrimers can also be captured with avidin/streptavidin handles for pull down experiments. Using avidin/streptavidin functionalized beads, the glycan dendrimers can be used to probe glycan binding molecules in flow cytometry setups and using a bifunctional amine-avidin linker the glycan dendrimers can be coupled to MagPlex® or MultiPlex® beads and adapted for Multiplex Luminex technology.

The commercial multivalent glycan polymers currently available are rather limited and restricted to “small” glycans (1 to 4 monosaccharides), limiting in-turn the experimental applications. The provided glycan dendrimer technology considerably increases the number of multivalent glycans polymers potentially available for a variety of applications including probing for carbohydrate binding molecules, inhibition assays, and glycan-induced cell signaling experiments. Homogenous purified glycans and heterogenous pool of glycans released from glycoproteins or complex tissues can be derivatized and coupled to a bifunctional, multivalent linker to create a diversity of multivalent glycan dendrimers.

In addition, the provided methodology offers the possibility to reconstitute the heterogeneity of a cell or tissue glycome on a multivalent, bifunctional dendrimer.

### Example 2: Generation of Glycan Dendrimers Using NHS/NH2 Coupling Chemistry

### Materials and Methods

Conjugation of Man5-/Man9-AEAB to 8-arm-PEG-NHS compound

The 8-arm-PEG-NHS (7 arms containing an NHS group, 1 arm containing a biotin group) (avg MW ˜21 kDa) was purchased from CreativePEGWorks as a custom compound. Man5- and Man9-AEAB (1 mg) were purchased from NatGlycan LLC.

500 μg of Man5-AEAB (˜360 nmol) or Man9-AEAB (˜240 nmol) were lyophilized in a 1.5 mL tube. 8-arm-PEG-NHS was resuspended at 21 mg/mL in 100 mM sodium phosphate, pH 8.5. 17.2 μL of the 8-arm-PEG-NHS solution (˜17.2 nmol) were added to the tube containing lyophilized the Man5-AEAB and 11.5 μL of the 8-arm-PEG-NHS solution (˜11.5 nmol) were added to the tube containing the lyophilized Man9-AEAB. The mixtures were incubated 3 h at RT with gentle rotation. The reactions were stopped, and any non-reacted NHS groups quenched, by the addition of 200 μL of 1M Tris, pH 7.5. The buffer of the dendrimer solution was exchanged with PBS using 5,000 MWCO Vivaspin 500 filter units (Sartorius). The glycan-dendrimers were finally resuspended in PBS at 1 mg/mL.

### Conjugation of Core1-Thr, Core2,3,4-Ser to 8-Arm-PEG-NHS Compound

Core1-Thr, Core2-Ser, Core3-Ser and Core4-Ser were purchased from Chemily Glycoscience as custom products.

350 nmol of Core1-T (˜550 μg), Core2-S (˜750 μg), Core3-S (˜580 μg) or Core4-S (˜780 μg) were lyophilized in a 1.5 mL tube. 8-arm-PEG-NHS was resuspended at 21 mg/mL in 100 mM sodium phosphate, pH 8.5. 50 μL of the 8-arm-PEG-NHS solution (˜50 nmol) were added to the tubes containing the lyophilized Core1-T and Core2,3,4-S and incubated 3 h at RT with gentle rotation. The reactions were then stopped, and any non-reacted NHS groups quenched, by the addition of 200 μL of 1M Tris, pH 7.5. The dendrimer solutions were finally buffer exchanged with PBS and using 5,000 MWCO Vivaspin 500 tubes (Sartorius). The glycan-dendrimers were finally resuspended in PBS at 1 mg/mL.

### Conjugation of GP100120 to 8-Arm-PEG-NHS Compound

The synthetic fibroin glycopeptide GP100120 (GAT*ES*RGAGAGA-NH₂, where S*=Ser*=GalNAcα-O-Ser, T*=Thr* =GalNAcα-O-Thr (SEQ ID NO:5) and including a C-terminal amide) was purchased from Sussex Research.

312 nmol of GP100120 (˜440 μg) were lyophilized in 1.5 mL tube. 8-arm-PEG-NHS was resuspended at 21 mg/mL in 100 mM sodium phosphate, pH 8.5. 15 μL of the the 8-arm-PEG-NHS solution (˜15 nmol) were added to the tubes containing the lyophilized GP100120 and incubated 3 h at RT with gentle rotation. The reactions were then stopped, and any non-reacted NHS groups quenched, by the addition of 200 μL of 1M Tris, pH 7.5. The dendrimer solutions were finally buffer exchanged with PBS and using 5,000 MWCO Vivaspin 500 tubes (Sartorius). The glycan-dendrimers were finally resuspended in PBS at 1 mg/mL.

### Lectin Assays of Dendrimers

To facilitate the detection of glycan-dendrimers, they were coupled via their biotin handle to streptavidin magnetic beads (MagnaBind™, Fisher Scientific). Magnetic beads separations and washes were performed with a DynaMag™—Spin Magnet (Fisher Scientific). 30 μL of beads were washed once with 200 μL of PBS, then resuspended in 44 μL of PBS before adding 6.3 μL of glycan-dendrimers at 1 mg/mL. The beads-dendrimers mixtures were incubated for 30-45′ at RT with gentle rotation. After removal of the solution, the beads were incubated with 3 drops of biotin solution (Streptavidin/Biotin Blocking Kit, Vector Laboratories) for 30′ at RT with gentle rotation. After removal of the biotin solution the beads were further blocked by incubation with 200 μL of TrisBufferSaline (TBS)+5% BSA, 30-45′ at RT with gentle rotation. After removal of TBS-5% BSA, the beads were washed once with PBS and resuspended in 50 μL PBS.

The lectins ConA, PNA, GSLII, VVL and AAL were purchased from Vector Laboratories as biotinylated proteins. The Avidin-HRP was purchased from SouthemBiotech. Lectins and Avidin-HRP were pre-complexed by incubating lectins (ConA at 0.5 μg/mL and all other lectins at 2 μg/mL) and Avidin-HRP (1:500 dilution) in TBS-1% BSA for 30-45′ at RT with gentle rotation. 3 μL of dendrimer beads were used per assay. The beads were incubated with 100 μL of the pre-complexed lectin-Avidin-HRP solution for 30-45′ at RT with gentle rotation. The beads were then washed 3× with 250 μL of TBS+0.1% tween 20. 100 μL of TMB (Abcam) were added to the beads and transferred to a 96-well plate for signal detection.

### Results

Additional glycan dendrimers were generated using 8-arm-PEG compounds carrying an N-hydroxysuccinimide (NHS) reacting group (instead of the alkyne group used above) (Creative PEGWorks). NHS reacts with primary amines to form covalent bonds in very simple reaction conditions.

Man5 and Man9 N-glycans tagged with AEAB (NatGlycan) to generate Man5- and Man9-dendrimers. The AEAB tag carries a primary amine, reacting with NHS in phosphate buffer pH 8.5 (FIGS. 3A-3C). The incorporation of Man5 or Man9 N-glycans was confirmed by selective reactivity to Concanavalin A lectin (ConA) and not to Peanut agglutinin lectin (PNA). Unmodified PEG-NHS dendrimer showed no signal to either lectin (FIG. 3D).

Using the same 8-arm-PEG-NHS compound, 0-glycan Core1-Thr, Core2,3,4-Ser glycan-amino acids (Chemily Glycoscience) were also coupled to generate Core1,2,3,4-dendrimers (FIGS. 4A-4B). Core1-T- and Core2-S-dendrimers were found positive by Peanut Agglutinin (PNA, specific for galactose as found in T-antigen) detection, and negative for both ConA and Griffonia Simplicifolia lectin II (GSLII, specific for terminal α- or β-linked N-acetylglucosamine) (FIG. 4C). Similarly, Core3-S- and Core4-S-dendrimers showed a moderate and high, respectively, reactivity to GSLII while being negative with PNA (FIG. 4D).

A synthetic glycopeptide containing 2 GalNac linked to 1 serine and 1 threonine (GP100120, Sussex Research) was conjugated to the same 8-arm-PEG-NHS compound (FIGS. 5A-5C). GP100120-dendrimers were found positive by Vicia Villosa lectin (VVL, specific for terminal N-acetylgalactosamine) detection and negative for PNA staining (FIG. 5D).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of identifying glycan-based binding interactions comprising contacting a target material with a known multivalent glycan scaffold comprising two or more glycans, and identifying element(s) from the target material that binds thereto.

2. The method of claim 1, wherein the target material is a population of cells comprising one more different cell types, or a cell lysate or other proteinaceous mixture comprising one or more glycan binding proteins (GBP).

3. The method of claim 2, wherein the target material is a population of cells comprising two more different cell types, or a cell lysate or other proteinaceous mixture comprising two or more glycan binding proteins (GBP).

4. The method of claim 3, wherein the target material is a population of cells and the target material element(s) is one or more of the cell types therein.

5. The method of claim 4, wherein the one or more cell types preferentially binds to the multivalent glycan scaffold compared to other cell type(s) in the population.

6. The method of any one of claims 2-5, further comprising isolating the multivalent scaffold with the cells bound thereto.

7. The method of any one of claims 2-6 further comprising characterizing the bound cells.

8. A composition comprising the glycan scaffold-bound cells of any one of claims 2-7.

9. The method of claim 2, wherein the target material is a cell lysate or other proteinaceous mixture and the target material element is one or more of the GBPs therein.

10. The method of claim 9, wherein the one or more GBPs preferentially binds to the multivalent glycan scaffold compared to other GBPs in the cell lysate or other proteinaceous mixture.

11. The method of any one of claims 9 or 10 further comprising characterizing the bound GBP.

12. The method of any one of claims 9-10 further comprising isolating the bound GBP.

13. A composition comprising the glycan scaffold-bound GBP of any one of claims 9-12.

14. A method of identifying glycan-based binding interactions comprising contacting one or more known target material elements with one or more species of multivalent glycan scaffolds each comprising two or more glycans, and identifying glycan scaffold(s) that bind thereto.

15. The method of claim 14 comprising contacting the one or more known target material elements with a library of different species of multivalent glycan scaffolds each comprising two or more glycans and identifying glycan scaffold(s) that bind thereto, optionally wherein the library comprises between about 2 to about 10,000 different species of glycan scaffolds, or any subrange or specific integer thereof, such as 2, 5, 10, 25, 50, 75, 100, 250, 500, 1,000.

16. The method of claim 14 or 15, wherein the known target material element is one or more cell types.

17. The method of claim 16, wherein one or more of the multivalent glycan scaffolds preferentially bind to the one or more cell types compared to other multivalent glycan scaffolds in the library.

18. The method of any one of claims 16 or 17 further comprising characterizing the bound multivalent glycan scaffold(s).

19. The method of any one of claims 16-18 further comprising isolating the multivalent glycan scaffold(s).

20. A composition comprising the glycan scaffold-bound cells of any one of claims 14-19.

21. The method of claims 14 or 15, wherein the known target material element is one or more GBP.

22. The method of claim 21, wherein one or more of the multivalent glycan scaffolds preferentially bind to the one or more GBP compared to other multivalent glycan scaffolds in the library.

23. The method of any one of claims 21 or 22 further comprising characterizing the bound multivalent glycan scaffold(s).

24. The method of any one of claims 21-23 further comprising isolating the multivalent glycan scaffold(s).

25. A composition comprising the glycan scaffold-bound GBP of any one of claims 21-24.

26. A method of identifying glycan-based binding interactions comprising contacting a target material with one or more unknown multivalent glycan scaffolds comprising two or more glycans, and identifying element(s) from the target material that binds thereto, optionally wherein the one or more unknown multivalent glycan scaffolds form a library comprising between about 2 about 10,000 different species of glycan scaffolds, or any subrange or specific integer thereof, such as 2, 5, 10, 25, 50, 75, 100, 250, 500, 1,000.

27. The method of claim 26, wherein the target material is a population of cells comprising one more different cell types, or a cell lysate or other proteinaceous mixture comprising one or more glycan binding proteins (GBP).

28. The method of claim 27, wherein the target material is a population of cells comprising two more different cell types, or a cell lysate or other proteinaceous mixture comprising two or more glycan binding proteins (GBP).

29. The method of claim 28, wherein the target material is a population of cells and the target material element(s) is one or more of the cell types therein.

30. The method of claim 29, wherein the one or more cell types preferentially binds to the multivalent glycan scaffold(s) compared to other cell type(s) in the population.

31. The method of any one of claims 26-30, further comprising isolating the multivalent scaffold(s) with the cells bound thereto.

32. The method of any one of claims 26-31 further comprising characterizing the bound cells and/or the glycan(s) bound thereto.

33. A composition comprising the glycan scaffold-bound cells of any one of claims 27-32.

34. The method of claim 27, wherein the target material is a cell lysate or other proteinaceous mixture and the target material element is one or more of the GBPs therein.

35. The method of claim 34, wherein the one or more GBPs preferentially binds to the multivalent glycan scaffold compared to other GBPs in the cell lysate or other proteinaceous mixture.

36. The method of any one of claims 34 or 35 further comprising characterizing the bound GBP and/or the glycan(s) bound thereto.

37. The method of any one of claims 34-36 further comprising isolating the bound GBP.

38. A composition comprising the glycan scaffold-bound GBP of any one of claims 34-37.

39. The method or composition of any one of claims 1-38, wherein the multivalent glycan scaffold comprises an underlying branched scaffold with two or more glycans and optionally one or more functional elements linked to the terminal ends thereof.

40. The method or composition of claim 39, wherein the underlying branched scaffold is a dendrimer.

41. The method or composition of claims 39 or 40, wherein the two or more glycans are all of the same species of glycan.

42. The method or composition of claims 39 or 40, wherein the two or more glycans are comprised of two or more different species of glycans.

43. The method or composition of any one of claims 39-42, wherein one or more of the functional element(s) is an agent that facilitates formation, imaging, localization, identification, isolation, and/or characterization of the glycan scaffold, and/or the target material element bound thereto.

44. The method or composition of any one of claims 39-43, wherein at least one of the functional elements is a nucleic acid comprising a barcode that specifically identifies the glycan scaffold.

45. The method or composition of any one of claims 39-44, wherein at least one of the functional elements is a fluorophore.

46. The method or composition of any one of claims 39-45, wherein the glycan is linked to 2-amino-N-(2-aminoethyl)-benzamide (AEAB) which is directly or indirectly linked to the terminal ends of the underling branched scaffold.

47. The method or composition of any one of claims 39-45, wherein at least one of the functional elements is an affinity tag.

48. The method of composition of any one of claims 39-47, wherein the glycan is linked to an amino acid which is directly or indirectly linked to the terminal ends of the underlying branched scaffold.

49. The method of claim 48, wherein the amino acid is no part of a polypeptide chain.

50. The method of claim 48, wherein the amino acid is part of a polypeptide chain.

51. The method of claim 50, wherein the polypeptide chain comprises two or more amino acid acids with glycans linked thereto.

52. The method of any one of claims 48-51, wherein the amino acid is serine or threonine.

53. The method or composition of any one of claims 1-52, wherein the glycan scaffold or target material element is immobilized, optionally to a surface or substrate such as bead, a slide, a plate, or a dish.

54. The method or composition of any one of claims 1-52, wherein neither the glycan scaffold or target material are immobilized.

55. The method or composition of claim 54, wherein the glycan scaffold and target material are in solution.

56. A method of treating a subject in need thereof, comprising identifying a glycan-based binding interaction according to any of the preceding methods and administering the subject a composition comprising an effective amount of a therapeutic multivalent glycan scaffold comprising the glycan of the identified interaction.

57. The method of claim 56, wherein therapeutic glycan induces or inhibits a cellular activity upon binding to cell or GBP of the interaction.

58. The method of claim 56, wherein therapeutic glycan comprise an active agent functionalized thereto.

59. The method of claim 58, wherein the active agent is a nucleic acid, small molecule, or polypeptide.

60. The method of claim 59, wherein the cell is a cancer cell and the active agent is an anti-cancer drug.

61. The method of claim 59, wherein the cell is an immune cell and the active agent is or encodes an antigen.

62. A composition comprising the therapeutic glycan of any one of claims 56-62.

63. A multivalent glycan scaffold comprising an underlying branched scaffold with two or more glycans and one or more functional elements linked to the terminal ends thereof.

64. The multivalent glycan scaffold of claim 63, wherein at least one of the functional element(s) is an agent that facilitates formation, imaging, localization, identification, isolation, and/or characterization of the glycan scaffold, and/or a target material element bound thereto.

65. The multivalent glycan scaffold of any one of claims 63 or 64, wherein at least one of the functional elements is a fluorophore.

66. The multivalent glycan scaffold of any one of claims 63-65, wherein the glycan is linked to 2-amino-N-(2-aminoethyl)-benzamide (AEAB) which is directly or indirectly linked to the terminal ends of the underling branched scaffold.

67. The multivalent glycan scaffold of any one of claims 63-66, wherein at least one of the functional elements is a nucleic acid comprising a barcode that specifically identifies the glycan scaffold.

68. The multivalent glycan scaffold of any one of claims 63-67, wherein at least one of the functional elements is an affinity tag.

69. The method of composition of any one of claims 63-68, wherein the glycan is linked to an amino acid which is directly or indirectly linked to the terminal ends of the underlying branched scaffold.

70. The method of claim 69, wherein the amino acid is no part of a polypeptide chain.

71. The method of claim 69, wherein the amino acid is part of a polypeptide chain.

72. The method of claim 71, wherein the polypeptide chain comprises two or more amino acid acids with glycans linked thereto.

73. The method of any one of claims 69-72, wherein the amino acid is serine or threonine.

74. The multivalent glycan scaffold of any one of claims 63-73, wherein the underlying branched scaffold is a dendrimer.

75. The multivalent glycan scaffold of any one of claim 63-74, wherein the underlying scaffold is formed of 2-16 arms, inclusive, optionally 8-16 arms.

76. The multivalent glycan scaffold of any one of claim 63-75, wherein the underlying scaffold and/or arms thereof is formed of polyethylene glycol (PEG) polymer.

77. The multivalent glycan scaffold of any one of claim 63-76 wherein the underlying scaffold is formed of 8-Arm PEG-Biotin/Alkyne, Biotin(1)/Alkyne(7), MW 20 k; Dendro Alkyne-PEG-Dendro Alkyne, PEG 10 k, G4 32 Alkyne; Dendro Alkyne-PEG-Dendro Alkyne, PEG 10 k, G5 64 Alkyne; or Dendro Alkyne-PEG-Dendro Alkyne, PEG 10 k, G6 128 Alkyne.

78. The multivalent glycan scaffold of any one of claims 63-77, wherein the multimeric glycan scaffold is between 10 kDa and 50kD, or between 20 kDa and 60 kDa, or between 20 kDa and 30 kDa, each inclusive, or any integer range thereof, or any specific integer therebetween.

79. The multivalent glycan scaffold of any one of claims 63-78, wherein the two or more glycans are all of the same species of glycan.

80. The multivalent glycan scaffold of any one of claims 63-79, wherein the two or more glycans are comprised of two or more different species of glycans.

81. The multivalent glycan scaffold of any one of claims 63-80 comprising the formula:
(Ligand)n:Scaffold:Detection/Separation Tag:Identifier Tag
wherein “Ligand” is present and is one or more species of glycan having n >1 copies (i.e., multivalent);
“Scaffold” is present and is the underlying branched scaffold, e.g., dendrimer or hyperbranched polymer;
one or both of “Detection/Separation Tag” and “Identifier Tag” are present and refer to the functional element e.g., (a) biotin or (HIS)6-8 peptide tag to facilitate separation/isolation, (b) a fluorophore to facilitate detection, (c) a unique DNA or RNA sequence (“barcode”) that can be PCR amplified and sequenced to facilitate identification; and/or (d) a drug or other material(s) as discussed elsewhere herein, e.g., nucleic acids such as DNA, RNA, iRNA, crispr guide RNA, etc., or vesicle containing a drug or such other materials that are intended to have a therapeutic or cell modifying effect or can include an antigen that is intended to induce (a) a protective immune response directed to a pathogen or an aberrant cell, e.g., tumor, that expresses the antigen or (b) immune tolerance to the antigen as a therapy for an autoimmune disorder directed against the antigen.

82. The multivalent glycan scaffold of any one of claims 63-81, wherein the glycan scaffold is not immobilized.

83. A composition comprising a plurality of multivalent glycan scaffold of any of claims 63-82 in solution.

84. The composition of claim 83, wherein the plurality of multivalent glycan scaffolds are not immobilized.


SEQUENCE LISTING (5 sequences)

SEQ ID NO: 1 - protein, 8 aa, synthetic construct
DYKDDDDK

SEQ ID NO: 2 - protein, 6 aa, synthetic construct
YPYDVP

SEQ ID NO: 3 - protein, 10 aa, synthetic construct
ILKKATAYIL

SEQ ID NO: 4 - protein, 10 aa, synthetic construct
EQKLISEEDL

SEQ ID NO: 5 - protein, 12 aa, synthetic construct
  CARBOHYD 3: note = O-linked (GalNAcAlpha)
  CARBOHYD 5: note = O-linked (GalNAcAlpha)
  SITE 12: note = alanine amide
GATESRGAGAGA